(19) United States
(12) Patent Application Publication
(10) Pub. No.: US 2026/0191882 A1
(43) Pub. Date: Jul. 9, 2026

(54) Orally Active, Brain Penetrant First-in-Class Small Molecule Midkine (MDK) Inhibitors for the Treatment of Malignancies and Non-Malignant Diseases

(71) Applicant: Hareesh B. Nair, San Antonio, TX (US)

(72) Inventor: Hareesh B. Nair, San Antonio, TX (US)

(21) Appl. No.: 19/442,815

(22) Filed: Jan. 7, 2026

Related U.S. Application Data
(60) Provisional application No. 63/743,099, filed on Jan. 8, 2025.

(51) Int. Cl. A61K 31/567; A61P 15/06; A61P 35/00; C07J 1/00

(52) U.S. Cl. CPC A61K 31/567; A61P 15/06; A61P 35/00; C07J 1/0096

(57) ABSTRACT

An orally active, brain penetrant first-in-class small molecule midkine inhibitor for use in the treatment of triple-negative breast cancer, brain tumors (including glioblastoma, medulloblastoma, and NF1-mutant optic pathway gliomas), ovarian and endometrial cancers, lung cancer, as well as gynecologic non-malignant conditions such as endometriosis, uterine fibroids, and preterm birth, in which tumor cells and/or pathological cells exhibit heightened midkine signaling and express multiple midkine receptors.

### CROSS REFERENCE TO RELATED APPLICATIONS

This original non-provisional application claims priority to and the benefit of U.S. provisional application Ser. No. 63/734,099, filed Jan. 8, 2025, and entitled “Orally active, brain penetrant first-in-class small molecule midkine (MDK) inhibitors for the treatment of malignancies and non-malignant diseases,” which is incorporated by reference herein.

### STATEMENT REGARDING FEDERALLY SPONSORED RESEARCH OR DEVELOPMENT

None.

### BACKGROUND OF THE INVENTION

### 1. Field of the Invention

The present invention relates to an orally active, brain penetrant small molecule for the treatment of malignancies and non-malignant diseases. More specifically, the present invention relates to an orally active, brain penetrant first-in-class small molecule midkine (MDK) inhibitor for use in the treatment of triple-negative breast cancer (TNBC), brain tumors, ovarian and endometrial cancers, lung cancer, as well as gynecologic non-malignant conditions in which tumor cells and/or pathological cells exhibit heightened MDK signaling and express multiple MDK receptors.

### 2. Description of the Related Art

Midkine (MDK) is a heparin-binding growth factor that acts as a cytokine regulating cell survival, migration, and proliferation. Emerging data demonstrate that MDK plays a critical role in the progression of Triple-negative breast cancer (TNBC). Elevated MDK expression is associated with metastasis, therapy resistance, and an overall more aggressive tumor phenotype. In addition, MDK modulates the tumor microenvironment by altering immune cell recruitment, cytokine secretion, and angiogenesis. Preclinical studies across multiple tumor types support MDK inhibition as a broad-spectrum therapeutic strategy. Consistent with these findings, MDK levels are increased in TNBC, and high MDK expression correlates with poor prognosis in TNBC patients.

MDK is a secreted, multifunctional extracellular protein that can act in a context-dependent manner as either a cytokine or a growth factor, thereby modulating multiple signaling pathways. The biological effects of MDK are dictated by the repertoire of MDK-binding receptors expressed in a given tissue. In cancer cells, several membrane-associated molecules have been identified as MDK receptors, including integrins, protein tyrosine phosphatase ζ (PTPζ), anaplastic lymphoma kinase (ALK), protein kinase C zeta (PKCζ), and the Notch2 receptor. In addition, MDK interacts with low-density lipoprotein receptor-related protein (LRP) and syndecans in other tissues. Through these receptors, MDK signaling has been linked to diverse biological processes, including mitogenic activity, inflammation, angiogenesis, metastasis, and stem cell self-renewal. Downstream, MDK influences canonical signaling modules such as PI3K and MAPK and transcriptional regulators such as NF-κB, Hes-1, and STAT family members. Functionally, MDK promotes temozolomide resistance in glioblastoma by enhancing cancer stem-like properties, and elevated serum MDK levels in endometrial cancer significantly correlate with poor prognosis and lymph node metastasis, supporting MDK as a promising therapeutic target. In breast cancer (BC), MDK drives tumor cell proliferation and migration via upregulation of NR3C1 and activation of the NF-κB pathway, and its overexpression is an independent adverse prognostic indicator for patient survival. The CD151-MDK axis has been shown to regulate the immune microenvironment in inflammatory BC, and increased serum MDK concentrations and tissue expression are associated with BC carcinogenesis. In TNBC cells, the deubiquitinase USP12 stabilizes MDK, thereby promoting angiogenesis and metastasis. The MDK receptor ALK is overexpressed in BC, with gene amplification reported in approximately 25% of TNBC cases, and an MDK-PKCζ-NFκB-p65 signaling cascade has been implicated in invasive progression. MDK-driven Notch2/Jak2-Stat3 signaling contributes to EMT, and STAT3 is recognized as a potential therapeutic node in TNBC. Moreover, MDK-PTPζ signaling plays a pivotal role in tumor progression, and PTPζ expression is an independent risk factor for TNBC recurrence and metastasis. Collectively, these data establish MDK signaling as a central driver of oncogenic processes, particularly in TNBC, and underscore a critical unmet need for an effective small-molecule inhibitor capable of blocking MDK-mediated oncogenic signaling. The reported receptors of midkine are shown in FIG. 1.

Rigor of Prior Research: Extensive published evidence supports the central hypothesis that MDK signaling is a key driver of cancer progression. MDK exerts pleiotropic effects and participates in multiple physiological processes. Under normal conditions, MDK expression is low in most tissues; however, its levels rise sharply in settings of inflammation and malignancy. In a wide range of solid tumors, MDK is markedly upregulated, and its signaling network contributes to tumor cell growth, metastatic dissemination, and resistance to conventional therapies. Tumors enhance MDK activity through both autocrine and paracrine loops. In addition, MDK profoundly shapes the tumor microenvironment. For example, MDK facilitates neutrophil trafficking in acute inflammation by increasing 32 integrin-mediated adhesion, and a CD74-dependent mechanism promotes B cell survival via MDK and RPTPζ. MDK-driven activation of CD8⁺ T cells has also been shown to establish a neuron-immune-cancer axis that sustains low-grade glioma. Taken together, these high-rigor studies demonstrate that both tumor-intrinsic and microenvironmental actions of MDK are critical to the progression of multiple malignancies, including breast cancer.

Given the central role of the MDK axis in malignancy, a number of groups have attempted to intervene upstream or downstream by targeting MDK receptors rather than MDK itself. For instance, inhibition of the MDK/ALK pathway with ALK inhibitors reduces tumorigenicity and chemoresistance in pancreatic ductal adenocarcinoma (PDAC) models. Metformin has been reported to counteract certain MDK-driven effects, including activation of PI3K and MAPK signaling in endometrial cancer. In hepatocellular carcinoma, shRNA-mediated MDK knockdown enhances the efficacy of anti-PD-1 immunotherapy by limiting the infiltration of immunosuppressive myeloid-derived suppressor cells (MDSCs). Similarly, treatment with functional anti-MDK antibodies suppressed the growth of osteosarcoma cell lines.

Reflecting the therapeutic interest in this pathway, a small biotechnology company is developing MDK-directed antibodies for clinical application, though these programs remain in early development (https://www.roquefortplc.com/). Despite this activity, no direct, ligand-based small-molecule inhibitors of MDK have been described. A compound termed iMDK has been reported to reduce MDK expression rather than directly bind MDK; iMDK inhibits lung adenocarcinoma growth and potentiates interferon-γ-mediated antitumor activity in ovarian cancer. While these data are encouraging, iMDK primarily lowers MDK levels and appears to act largely through INK, rather than functioning as a direct MDK antagonist. Likewise, MDK “inhibitors” discovered via promoter-reporter assays act on upstream regulatory elements, not MDK itself, and tumor cells may rapidly escape such agents by activating alternative pathways to sustain MDK expression. Thus, there is a need for a small molecule MDK inhibitor that acts on MDK itself.

It is an object of the present invention to have a small molecule that directly blocks MDK action or signaling that yields the full realization of MDK as a therapeutic target.

To date, the lack of a direct, small-molecule MDK antagonist has impeded clinical translation of this biology. HBS-101 and its analogues uniquely fill this gap and provide a platform of therapeutic agents for targeted intervention in MDK-driven malignancies and non-malignant MDK-mediated disorders.

### BRIEF SUMMARY OF THE INVENTION

The present invention comprises small-molecule MDK inhibitors, exemplified by HBS-101 and its analogues, for use in the treatment of MDK overexpressing tumors (breast, endometrial, pancreatic, ovarian, liver) as well as triple-negative breast cancer (TNBC), brain tumors (including glioblastoma, medulloblastoma, and cortical tumors associated with neurofibromatosis NF1-mutant optic pathway gliomas (NF1-OPG)), ovarian and endometrial cancers, lung cancer, as well as gynecologic non-malignant conditions and diseases involving inflammation including pericarditis, other cardiac inflammation, endometriosis, polycystic ovary syndrome (PCOS), uterine leiomyomas and fibroids, and preterm birth, in which tumor cells and/or pathological cells exhibit heightened MDK signaling and express multiple MDK receptors. The MDK inhibitors of the present invention abrogate fibrotic lesions in kidney and liver, block progression to carcinoma. The MDK inhibitors of the present invention further effectively block the interaction of PKCzeta, Syndycans, Notch2, ALK and LRP1 or integrins to its receptors and also blocks downstream targets such as STAT3 and NFkB. The antiproliferative compounds of the present invention may be used as a biomarker/companion diagnostic (CDx) for selecting suitable population to treat with the MDK small molecule inhibitors of the present invention to down-regulate phosphorylation of STAT3 and inhibit NFkB signaling.

The MDK inhibitor compounds of the present invention inhibit lung inflammation and lung fibrosis. The MDK small molecule inhibitor compounds synergistically act with gemcitabine and other chemotherapeutic agents and immune checkpoint inhibitors.

The structures of the midkine inhibitor of the present invention are as follows:

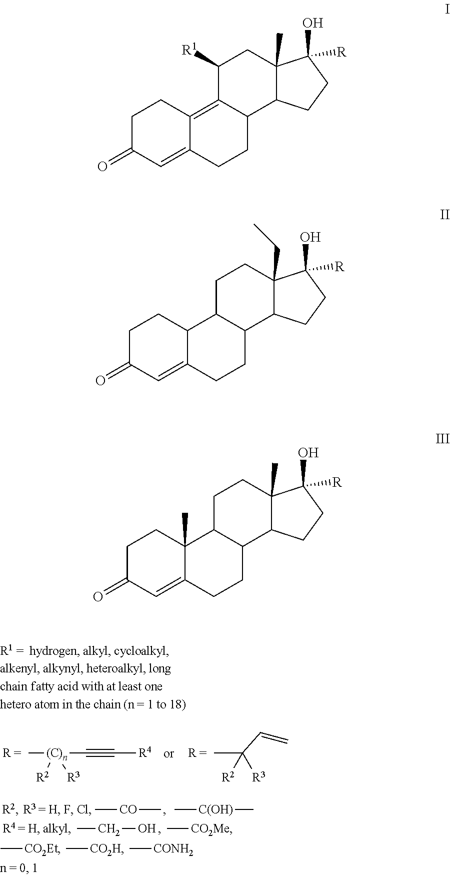

### Structures of Midkine Inhibitor

where R¹ is H, alkyl, cycloalkyl, alkenyl or a long chain fatty acid or any long chain with at least one hetero atom and R² and R³ are H, F, Cl, —CO—, —C(OH)—, and R⁴ is H, alkyl hydroxy alkyl, methyl or ethyl ester, acid or amide.

Other embodiments or variations of the midkine inhibitor of the present invention include the following:

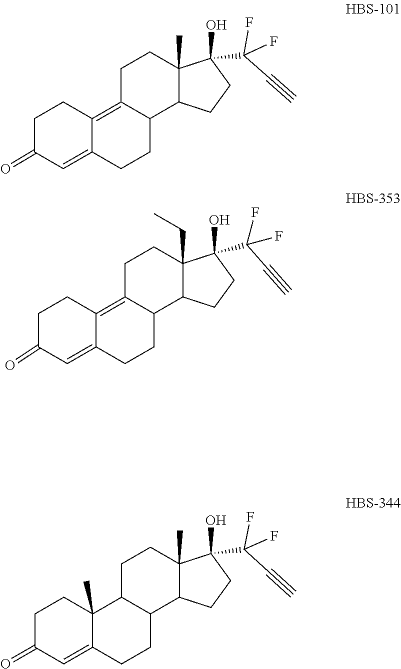 [SMILES: C#CC(F)(F)[C@]1(O)CCC2C3CCC4=CC(=O)CCC4=C3CC[C@@]21C ; C#CC(F)(F)[C@]1(O)CCC2C3CCC4=CC(=O)CCC4=C3CC[C@@]21CC ; C#CC(F)(F)[C@]1(O)CCC2C3CCC4=CC(=O)CC[C@]4(C)C3CC[C@@]21C]

By directly binding to and inhibiting MDK, HBS-101 and related compounds are configured to simultaneously block MDK-driven signaling across diverse receptor complexes, providing a unified and potentially more cost-effective strategy compared with approaches that individually target downstream MDK receptors. The small molecule MDK inhibitors of the present invention robustly inhibit cancer cell proliferation, trigger apoptosis, reduce stem-like/tumor-initiating phenotypes, demonstrate in vivo antitumor efficacy, and favorably remodel the tumor microenvironment. Consistent with these findings, genetic ablation or knockdown of MDK in the above-mentioned disease models leads to marked reductions in cell viability and clonogenic growth, confirming MDK as a critical functional driver.

The discovery and development of a first-in-class, direct small-molecule inhibitor of MDK provides a clinically meaningful advance over existing MDK-targeted strategies. Antibody-based approaches to MDK inhibition are costly to manufacture and administer, limiting access and imposing a substantial financial burden on healthcare systems. In contrast, a low-cost, orally available small-molecule agent that selectively targets MDK has the potential to deliver comparable or superior therapeutic benefit while significantly reducing overall treatment costs. At present, the absence of a small molecule that directly blocks MDK signaling constitutes a major scientific and translational gap and has hindered the full realization of MDK as a therapeutic target.

The present invention provides a rationally designed lead compound, HBS-101, and structurally related analogues that bind directly to MDK and function as MDK inhibitors. HBS-101 and its analogues offer a novel and effective approach to neutralizing MDK-driven oncogenic activity. Without being bound by theory, these compounds inhibit the interaction of MDK with multiple MDK receptors commonly upregulated in TNBC and other MDK-dependent cancers, including integrins, PTPζ, ALK, and the Notch2 receptor. In preclinical studies described herein, HBS-101 treatment led to suppression of MDK-dependent signaling pathways, including AKT, NFκB, mTOR, and STAT3, and resulted in reduced cancer cell survival. The relatively simple and scalable synthetic route, chemical stability, and compatibility with oral dosing make HBS-101 and its analogues attractive candidates for clinical development in patients with the aforementioned malignancies and MDK-driven diseases.

### BRIEF DESCRIPTION OF THE SEVERAL VIEWS OF THE DRAWINGS

FIG. 1 shows reported receptors of midkine affected by the midkine inhibitors of an embodiment of the present invention.

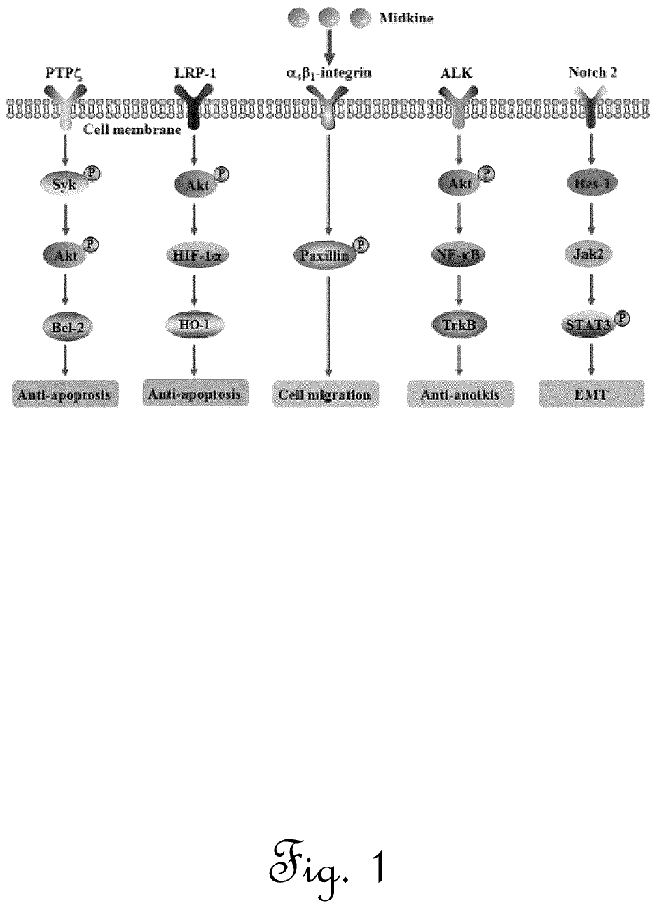

FIG. 2 depicts a graph confirming high affinity bonding of a midkine inhibitor of an embodiment of the present invention to midkine.

FIG. 3 depicts Western blotting showing the effectiveness of a midkine inhibitor of an embodiment of the present invention.

FIG. 4 depicts Western blotting showing comparison of vehicle with a midkine inhibitor of an embodiment of the present invention.

FIG. 5 shows a graphical representation of the relative band intensity versus temperature for a vehicle control and an embodiment of a midkine inhibitor of the present invention for a patient-derived glioblastoma stem cell.

FIG. 6 shows a graphical representation of the relative PREluc activity versus a vehicle control and embodiments of midkine inhibitors of the present invention for a patient-derived glioblastoma stem cell.

FIG. 7 shows transient MDK knockdown with three independent siRNAs in MDA-MB-231 cells with reduced cell viability compared with scramble control, confirmed by decreased MDK protein levels.

FIG. 8 shows transient MDK knockdown with three independent siRNAs in MDA-MB-231 cells with reduced cell viability compared with scramble control, confirmed by decreased MDK protein levels.

FIG. 9 depicts Western blotting showing transient MDK knockdown with three independent siRNAs in MDA-MB-231 cells showing decreased MDK protein levels.

FIG. 10 depicts transient MDK knockdown with three independent siRNAs in MDA-MB-231 cells showing decreased MDK protein levels.

FIG. 11 depicts two independent CRISPR-derived MDK knockout clones (KO-1, KO-2) showing loss of MDK expression.

FIG. 12 depicts two independent CRISPR-derived MDK knockout clones (KO-1, KO-2) exhibiting a strong reduction in clonogenic capacity relative to wild-type cells.

FIG. 13 is a graphical representation of percentage of colonies versus two independent CRISPR-derived MDK knockout clones (KO-1, KO-2) exhibiting a strong reduction in clonogenic capacity relative to wild-type cells.

FIG. 14 is a graphical representation of percentage of cell viability versus dosage of HBS-101 across a panel of TNBC lines showing reduction in cell viability.

FIG. 15 is a graphical representation of percentage of cell viability versus dosage of HBS-101 in representative MDA-MB-231 and BT-549 cells, where HBS-101 produced a marked, dose-dependent suppression of colony formation.

FIG. 16 shows representative MDA-MB-231 and BT-549 cells, where HBS-101 produced a marked, dose-dependent suppression of colony formation with near-complete loss of colonies at ≥5-10 μM.

FIG. 17 is a graphical representations of treatment of MDA-MB-231 and BT-549 cells with HBS-101 which resulted in a marked increase in Annexin V-positive cells compared with vehicle-treated controls, indicating strong induction of programmed cell death in both TNBC models.

FIG. 18 is a graphical representation of tumor volume versus days of treatment for a MDA-MB-231 xenograft model showing pronounced, dose-dependent inhibition of tumor growth compared with vehicle-treated mice, as evidenced by reduced tumor volumes over time.

FIG. 19 is a graphical representation of tumor volume versus days of treatment for a MDA-MB-231 xenograft model showing pronounced, dose-dependent inhibition of tumor growth compared with vehicle-treated mice, as evidenced by significantly lower terminal tumor.

FIG. 20 is a graphical representation of tumor volume versus days of treatment for a MDA-MB-231 xenograft model showing stable body weights throughout the treatment.

FIG. 21 depicts several tumors where reduction in tumor size is more pronounced at the higher HBS-101 dosage, illustrating the dose-dependent antitumor activity reduction effect.

FIG. 22 shows a graphical representation of plasma concentration over time of single-dose PK evaluation of HBS-101 in rats with a terminal plasma half-life of approximately 1.6 hours.

FIG. 23 is a graphical representation of total flux versus days after implantation of tumors in an orthotopic MDA-MB-231-BrM GFP-Luc model following treatment using an MDK inhibitor of the present invention.

FIG. 24 is a graphical representation of the probability of survival days after implantation of tumors following treatment in an orthotopic MDA-MB-231-BrM GFP-Luc model using an MDK inhibitor of the present invention.

FIG. 25 depicts representative tumor images of the subject mice comparing the tumor treated with vehicle and the tumor treated with HBS-101 of the present invention.

FIG. 26 shows an array of MDA-MB-231 cells exposed to and treated with a matrix of either HBS-101 or doxorubicin or a combination of HBS-101 and doxorubicin concentrations for a time certain at various doses, followed by a 10-day colony formation assay.

FIG. 27 depicts a graphical representation of a quantitative synergy analysis with respect to FIG. 26 using commercially available software, confirming HBS-101 and doxorubicin cooperate to enhance anti-tumor activity in TNBC cells.

FIG. 28 depicts a Western blotting of HCC-70 model cells treated with HBS-101, the midkine inhibitor of the present invention, and measuring the treatment effect on expression of MDK receptors, MDK downstream signaling and EMT genes.

FIG. 29 is a graphical representation of HCC-70 model cells treated with HBS-101 illustrating a significant downregulation of a panel of MDK-responsive genes involved in invasion, survival, and inflammation.

FIG. 30 depicts Western blotting illustrating that, consistent with direct pathway inhibition, the midkine inhibitor of the present invention diminished activation of downstream effectors.

FIG. 31 depicts Western blotting illustrating that the midkine inhibitor of the present invention also reverses EMT-associated changes, increasing E-cadherin while reducing MMP-9 and various transcription factors, indicating that MDK blockade by the midkine inhibitor of the present invention shifts TNBC cells toward a less invasive, more epithelial phenotype.

FIG. 32 depicts a graphical representation of the effect of HBS-101 on 4T1 TNBC xenograft tumor volume over days of treatment.

FIG. 33 depicts a graphical representation of the effect of HBS-101 on 4T1 TNBC xenograft body weight over days of treatment.

FIG. 34 depicts a graphical representation of the effect of HBS-101 on 4T1 TNBC xenograft tumor weight.

FIG. 35 depicts a graphical representation of the effect of HBS-101 on 4T1 TNBC xenograft and status of NK cells.

FIG. 36 is a graphical representation of concentration over time of the brain concentration of the midkine inhibitor of the present invention.

FIG. 37 is a graphical representation of an orthotopic patient-derived GSC 082209 glioblastoma model illustrating HBS-101 treatment significantly reducing intracranial tumor burden as measured by bioluminescent flux.

FIG. 38 is a graphical representation of probability of survival over days of implantation of tumor in an orthotopic patient-derived GSC 082209 glioblastoma model illustrating a marked improvement in overall survival of mice treated with the midkine inhibitor of the present invention as compared with vehicle-treated mice.

FIG. 39 is a graphical representation of probability of survival over days of implantation of tumor in a second independent GBM model (3422-fPPP) where HBS-101 extended survival relative to control.

FIG. 40 shows a graphical representation of the probability of survival against days after implantation is shown of an orthotopic GSC 040815 glioblastoma model showing treatment with either HBS-101 or temozolomide (TMZ) or both significantly prolonged survival relative to vehicle controls.

FIG. 41 shows a graphical representation of the treatment with the MDK-modulating agent completely preventing preterm birth in all control dams preterm delivery as compared to the treated dams where none delivered preterm.

FIG. 42 is a graphical representation of the percentage of pregnancy over time in days, illustrating the mice receiving MDK-modulating treatment-maintained pregnancy significantly longer than vehicle controls.

FIG. 43 is a graphical representation of the percentage of cell viability over dosage of the midkine inhibitor of the present invention demonstrating the effect of HBS-101 on the viability of normal HFTEC and OCa cell line TOV21G, and primary OCa cell lines as determined by the MTT assay.

FIG. 44 is a graphical representation of the percentage of cell viability over dosage of the midkine inhibitor of the present invention demonstrating the effect of HBS-101 on the viability of established ovarian cancer cell lines, as determined by the MTT assay.

FIG. 45 is a graphical representation of the percentage of cell viability over dosage of the midkine inhibitor of the present invention demonstrating the effect of HBS-101 on the viability of ascites cells, as determined by the MTT assay.

FIG. 46 is a graphical representation of the percentage of cell viability over dosage of the midkine inhibitor of the present invention demonstrating the effect of HBS-101 on the viability of primary OCa cells, as determined by the MTT assay.

FIG. 47 is a graphical representation of the percentage of cell viability over dosage of the midkine inhibitor of the present invention illustrating a direct comparison with modulator iMDK where HBS-101 achieves greater growth inhibition at lower concentrations, demonstrating superior efficacy as an MDK-targeted small-molecule agent.

FIG. 48 depicts a graphical representation of the tumor volume over days of treatment with the midkine inhibitor of the present invention in an ovarian cancer xenograft model showing marked reduction in tumor volume of patient derived OCa-67 xenografts bearing mice treated orally with HBS-101 as compared with vehicle.

FIG. 49 depicts a graphical representation of tumor weight demonstrating tumors from HBS-101-treated animals were significantly lighter than those from control mice.

FIG. 50 depicts a graphical representation of body weight demonstrating comparable body weights between groups over the course of treatment.

FIG. 51 depicts a graphical representation of tumor weight over days with HBS-101 and carboplatin as compared with vehicle in an EC98 endometrial cancer PDX model, illustrating the combination regimen of HBS-101 and carboplatin produced the greatest antitumor effect.

FIG. 52 depicts a graphical representation of tumor weight over days with HBS-101 and carboplatin as compared with vehicle in an EC98 endometrial cancer PDX model, illustrating the combination regimen of HBS-101 and carboplatin produced the smallest terminal tumor weights.

FIG. 53 depicts a graphical representation of tumor weight over days with HBS-101 and carboplatin as compared with vehicle in an EC98 endometrial cancer PDX model, illustrating the combination regimen of HBS-101 and carboplatin maintained stable body weight comparable to single-agent and vehicle groups.

FIG. 54 a graphical representation of tumor cross-sectional area over time in a SW1573 NSCLC xenograft model where HBS-101 markedly slowed tumor progression compared with vehicle-treated controls, and higher HBS-101 dosages were more effective than lower HBS-101 dosages.

FIG. 55 depicts a modeled structure of MDK, the interaction sites therein, and the number of contacts maintained over time.

### DETAILED DESCRIPTION OF THE INVENTION

The inhibitor compounds of the present invention showed the following advantages, described below.

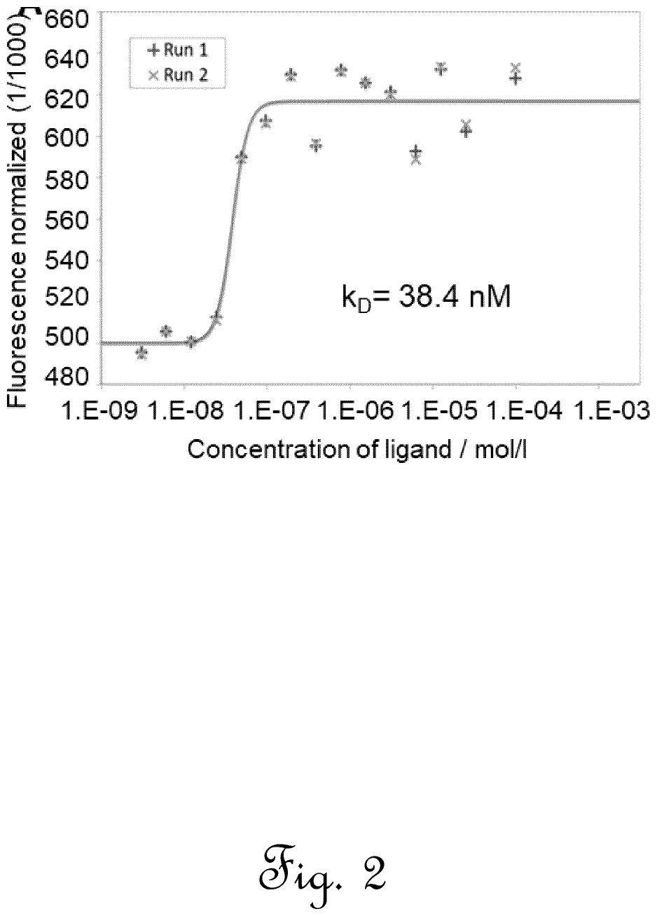

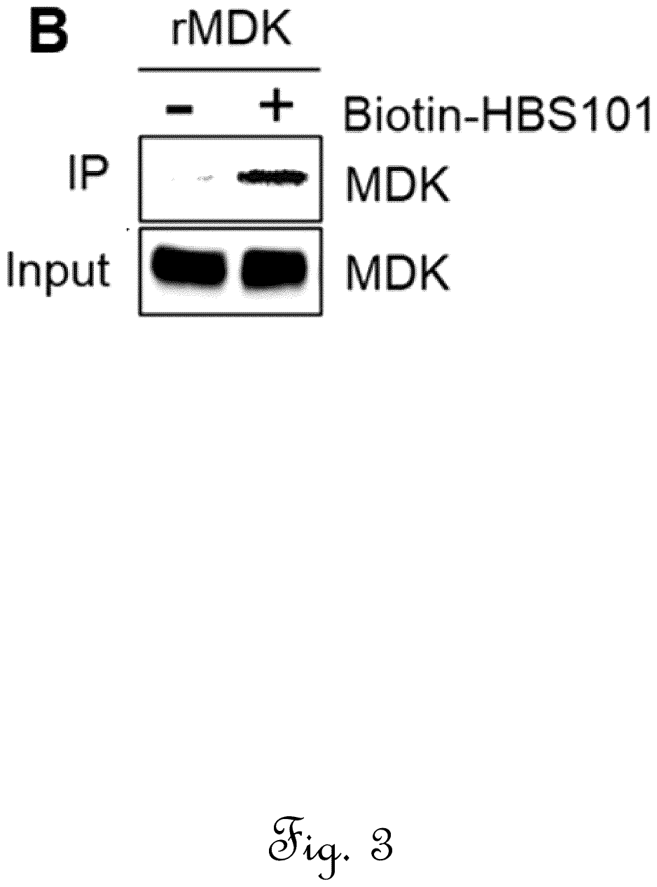

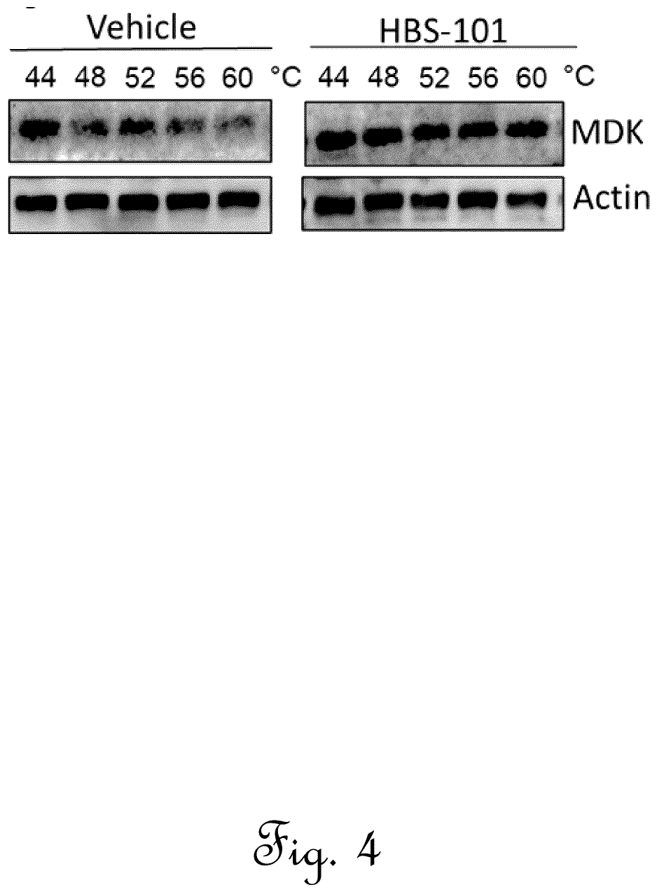

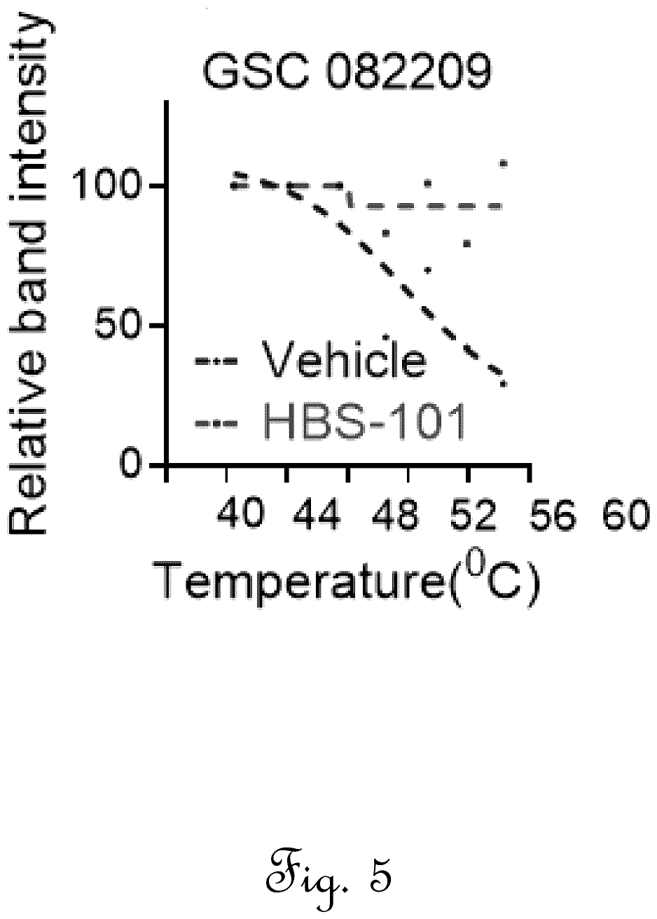

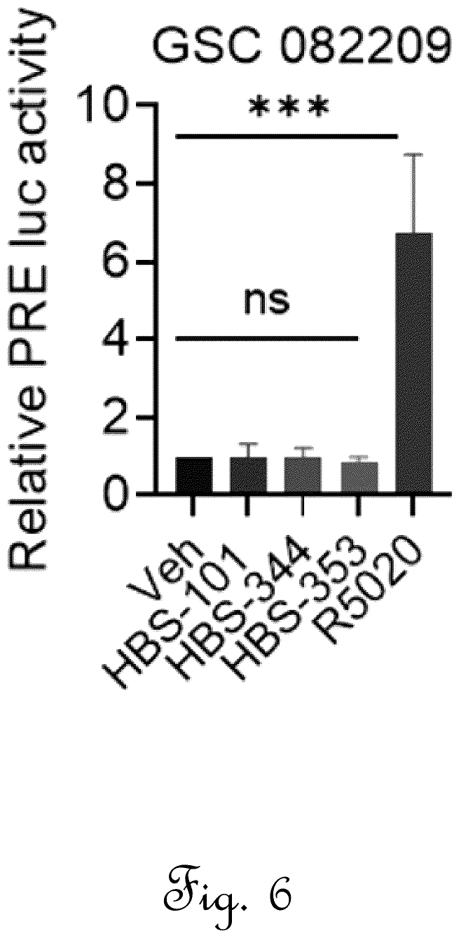

Microscale thermophoresis (MST) and Cellular thermal shift assays (CETSA) confirmed direct binding of HBS-101 to MDK: MST assays using recombinant protein. Referring to FIGS. 2-4, binding of rMDK protein to HBS-101 was determined using MST assay. MDK (rMDK) confirmed high affinity binding of HBS-101 to MDK (38.4 nM), as shown in FIG. 2. Referring now to FIG. 3, Biotin-tagged HBS-101 (5 μM) was incubated with purified MDK protein (10 ng) and subjected to streptavidin pulldown which confirmed the direct interaction of HBS-101 with MDK. Cellular thermal shift assay (CETSA) was then used. CETSA is a biophysical method to monitor drug target engagement in cells by measuring changes in protein thermal stability upon ligand binding. This assay provides insights into drug binding and target engagement in a cellular context. Referring to FIG. 4, GSC082209 cells were treated with dimethyl sulfoxide (DMSO) or HBS-101 (5 μM) for 6 hours and lysates were heated for 3 min at 33 to 60° C., using 4° C. intervals. Western blotting shows the degradation of MDK in vehicle control but not in HBS-101 treatment, as shown in FIG. 4, confirming the binding of HBS-101 to MDK. HSB-101 interaction with MDK was confirmed using Western blotting (FIG. 4). CETSA results provided further evidence that HBS-101 binds directly to MDK in patient-derived glioblastoma stem cells (GSCs). Importantly, quantitation of band intensities was shown, as seen in FIG. 5. To further check whether HBS-101 is a specific inhibitor of MDK and does not retain progesterone agonist activity (since HBS-101 has steroid scaffold), GSCs that stably express progesterone receptor reporter (PGRluc) were treated with HBS analogs (HBS-101, HBS-353, HBS-344) and progesterone agonist (R5020), as shown in FIG. 6. Still referring to FIG. 6, the effect of HBS-101 and other HBS analogs HBS-353, HBS-344 (10 μM, 6 h treatment) on PGR-luc reporter activity was determined. Progesterone agonist (R5020) was used as a positive control. ***p<0.001. Analysis of Variance (ANOVA). The results obtained confirmed that HBS-101 and other HBS analogs did not exhibit progesterone-like agonist activity. Altogether, these results confirm that HBS-101 directly interact with MDK and a selective MDK inhibitor.

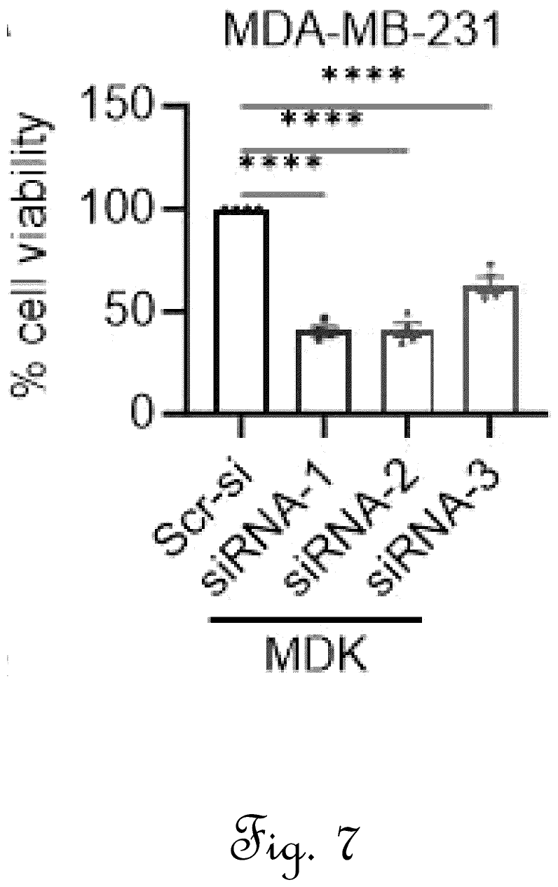

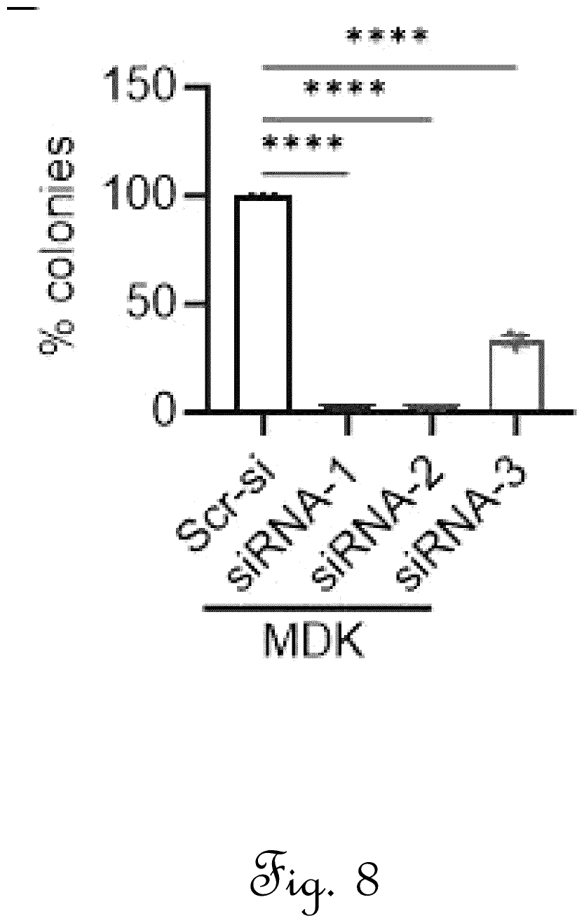

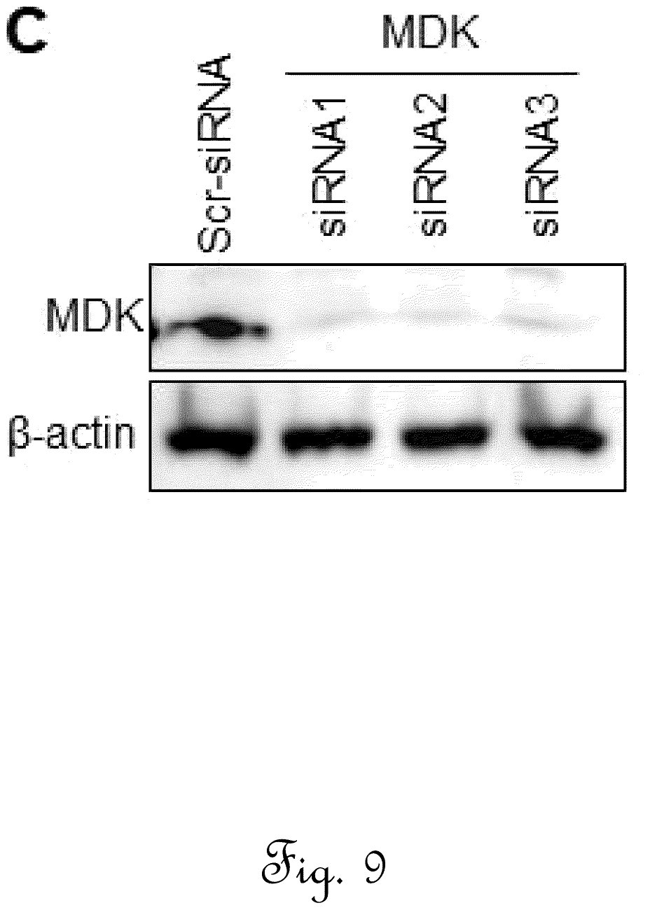

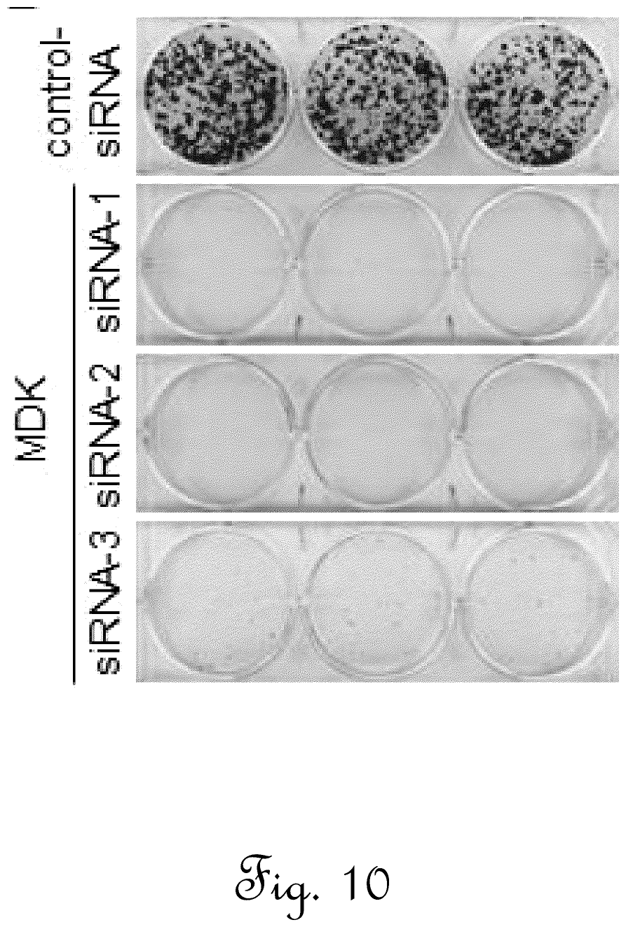

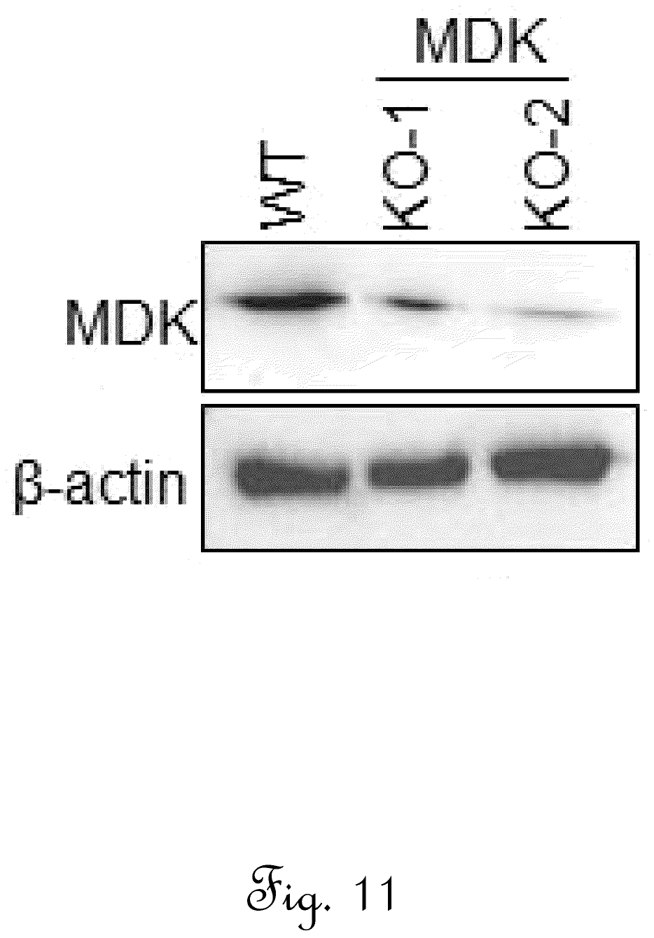

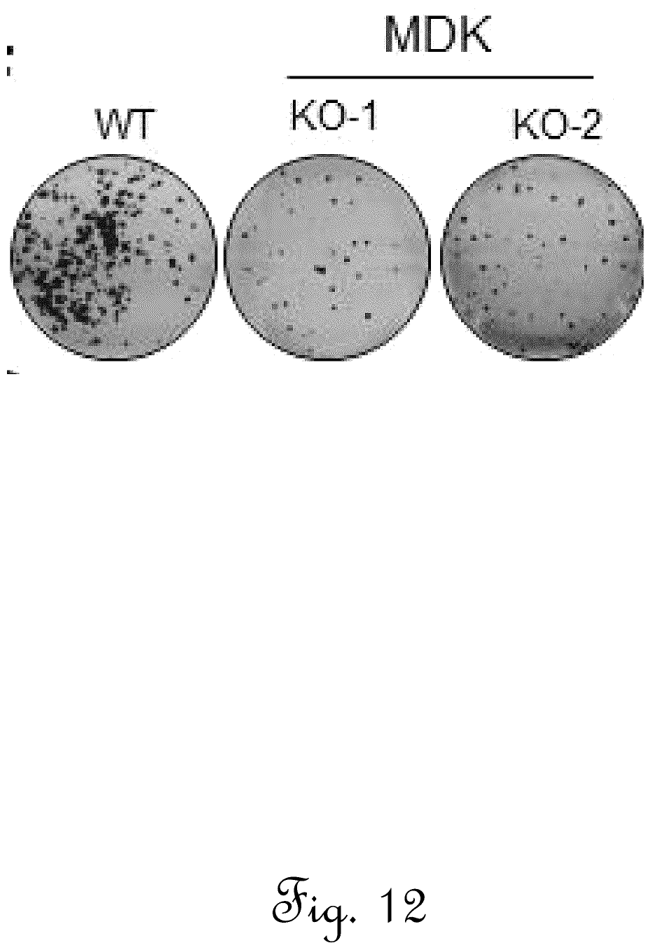

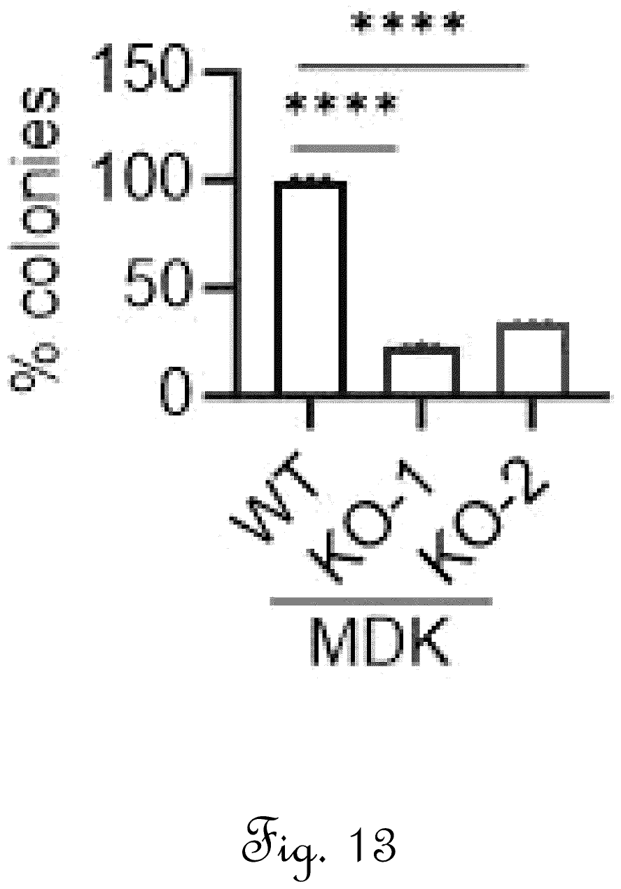

Genetic suppression of MDK markedly impaired TNBC cell survival and clonogenic growth. Referring now to FIGS. 7-13, transient MDK knockdown with three independent small interfering ribonucleic acids (siRNAs) in MD Anderson-Metastatic Breast-231 (MDA-MB-231) cells reduced cell viability and colony formation compared with scramble control, as confirmed by decreased MDK protein levels (FIGS. 7-10). Similarly, two independent clustered interspaced short palindromic repeats (CRISPR)-derived MDK knockout clones (KO-1, KO-2) showed loss of MDK expression (FIG. 11) and exhibited a strong reduction in clonogenic capacity relative to wild-type cells (FIGS. 12-13). These data establish MDK as a critical survival and growth driver in TNBC cells ****p<0.0001.

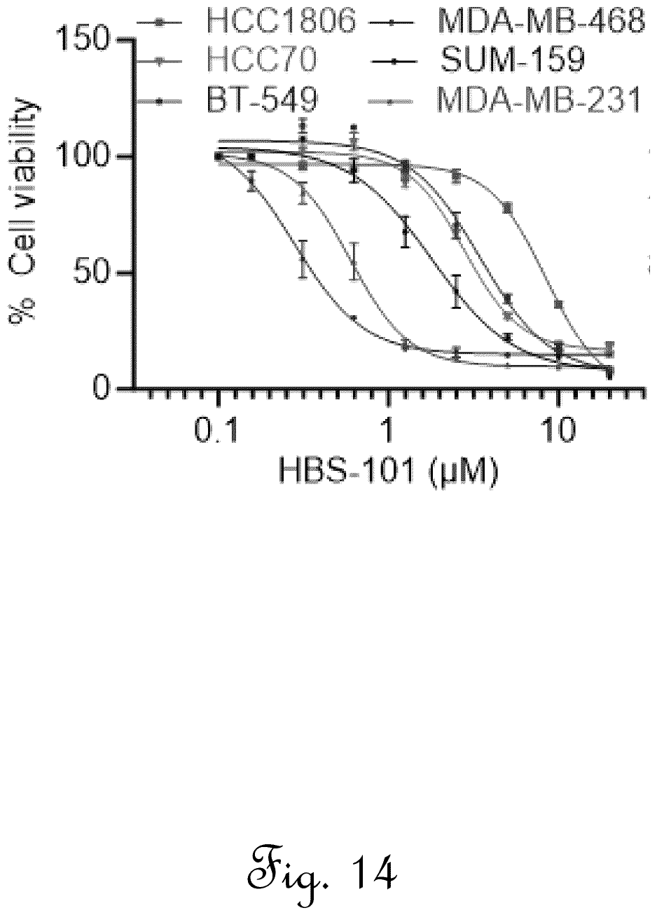

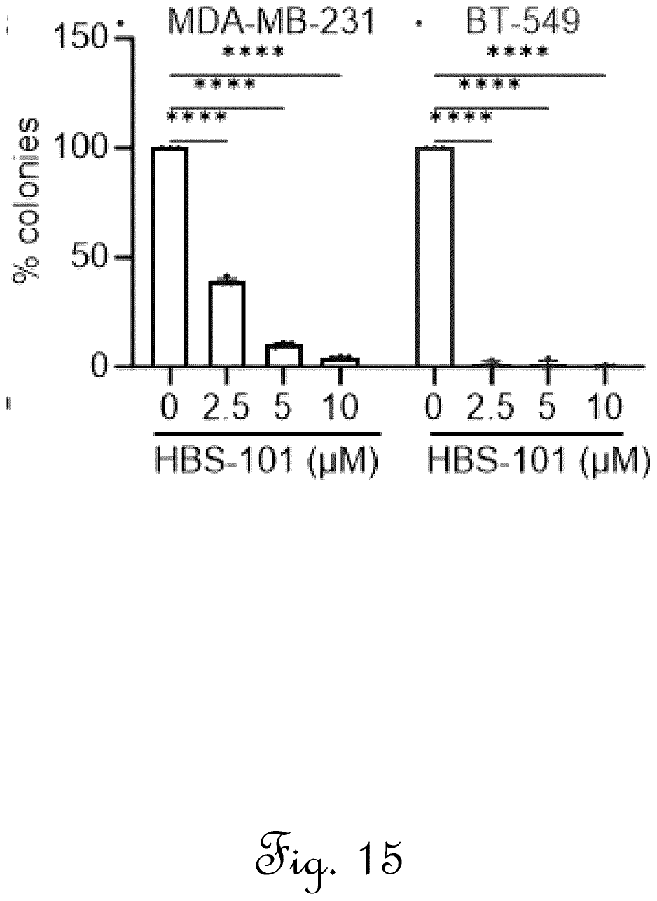

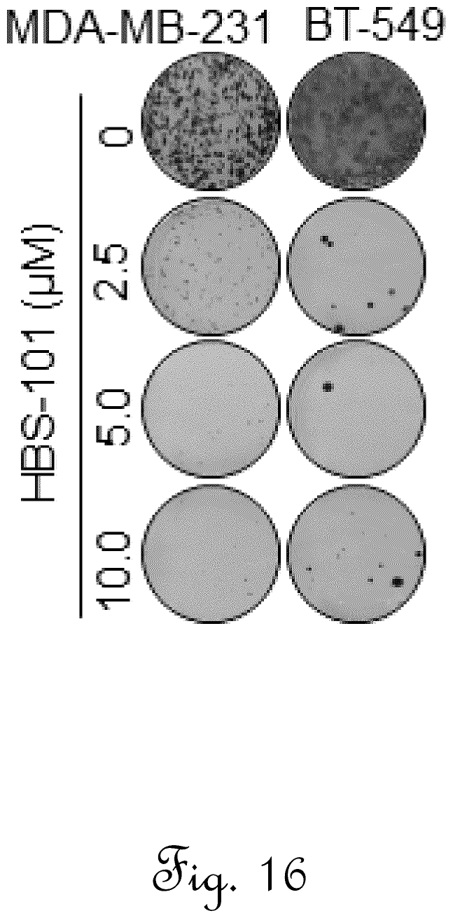

HBS-101 potently inhibits growth and clonogenic survival of multiple TNBC cell lines. Dose-response studies showed that HBS-101 reduced cell viability across a panel of TNBC lines (HCC1806, HCC70, BT-549, MDA-MB-468, SUM-159, and MDA-MB-231) with sub-to low-micromolar potency, as shown in FIG. 14. In representative MDA-MB-231 and BT-549 cells, HBS-101 produced a marked, dose-dependent suppression of colony formation, with near-complete loss of colonies at ≥5-10 μM, as shown in FIGS. 15 and 16. These data demonstrate that HBS-101 exerts broad and robust antiproliferative activity in TNBC models.

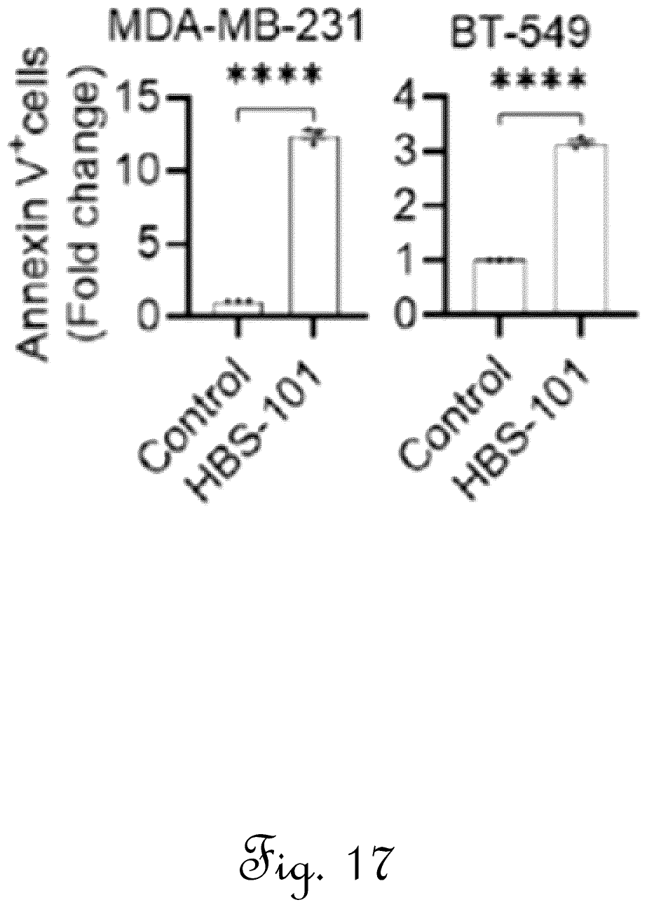

HBS-101 induces robust apoptotic cell death in TNBC cells. Turning now to FIG. 17, Annexin V Assay was utilized to evaluate the impact of HBS-101 (20 μM) at 24 hrs. on the induction of apoptosis in MDA-MB-231 and BT-54 TNBC model cells. ****p<0.0001. Treatment of MDA-MB-231 and BT-549 cells with HBS-101 (20 μM, 24 h) resulted in a marked increase in Annexin V-positive cells compared with vehicle-treated controls, as shown in FIG. 17. This is indicative of strong induction of programmed cell death in both TNBC models (****p<0.0001).

Oral HBS-101 Produces Dose-Dependent Antitumor Activity in TNBC Xenografts Without Overt Toxicity. In a MDA-MB-231 xenograft model, MDA-MB-231 tumor pieces were implanted orthotopically into nude mice. These MDA-MB-231 (n=7) xenografts bearing mice were treated orally with vehicle or HBS-101 (2 mg/kg/day and 5 mg/kg/day). Treatment started after 10 days of tumor implantation. The treatment consisted of HBS-101 at 2 mg/kg and 5 mg/kg administered 5 oral doses per week in 30% captisol as vehicle. The mice were sacrificed after 22 doses of HBS-101.

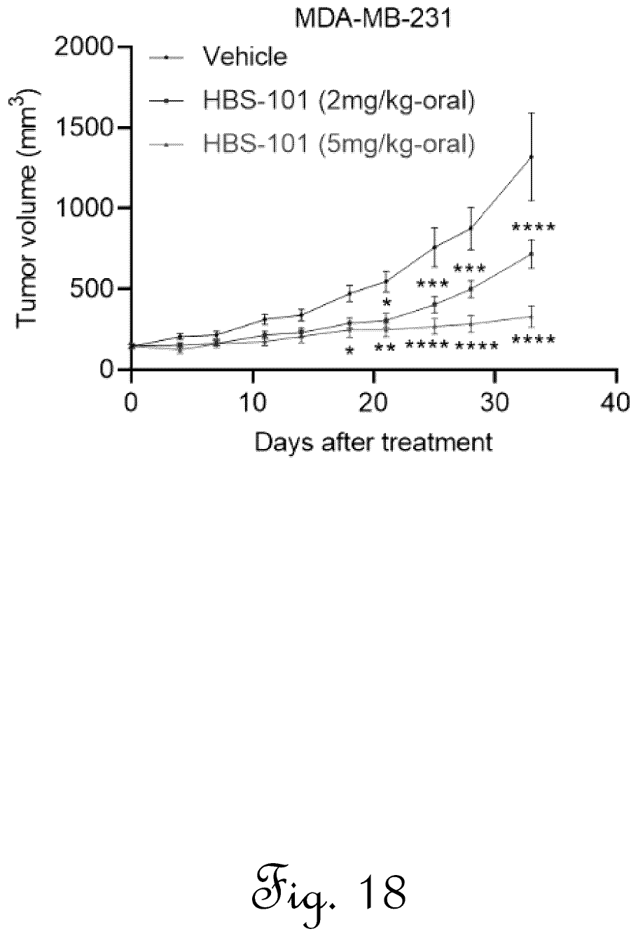

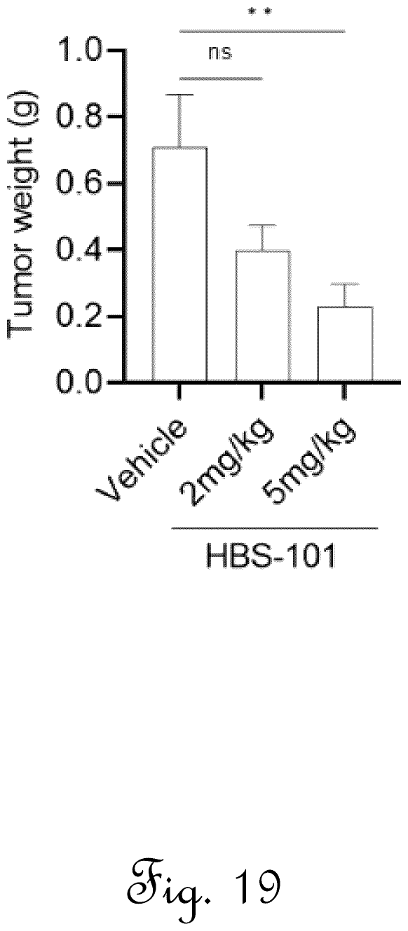

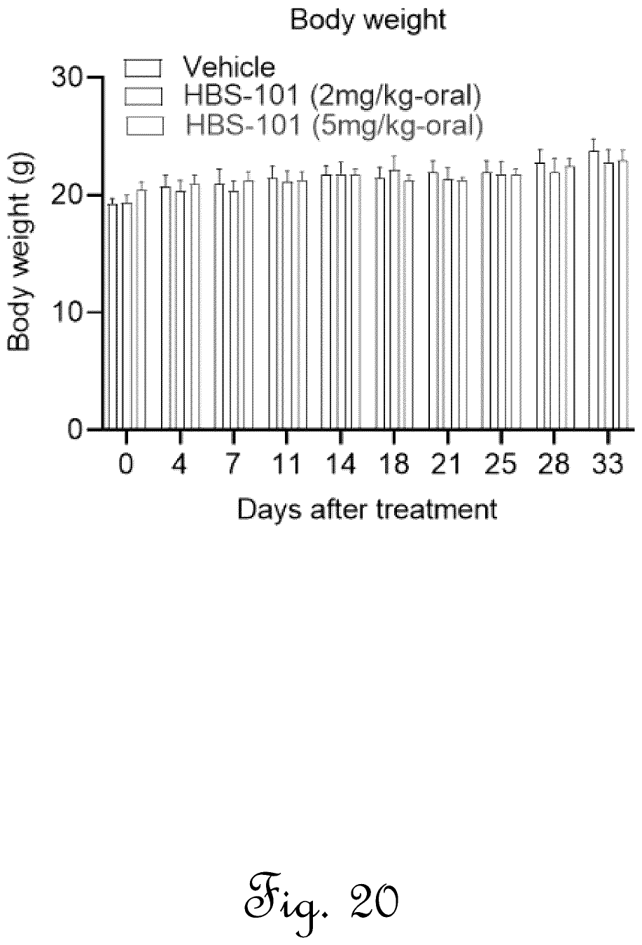

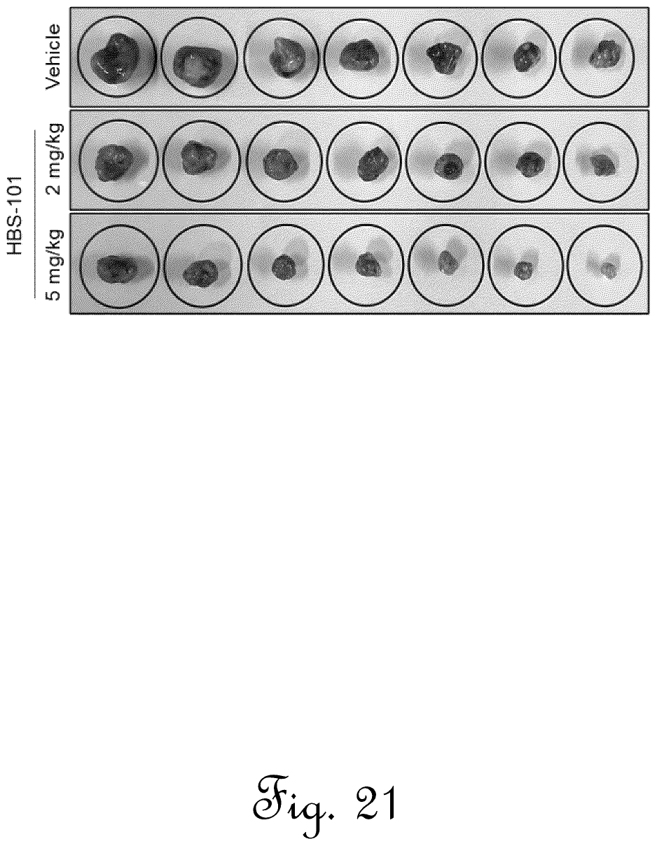

Tumor volumes (as well as tumor weights and body weights) were measured twice a week. Referring now to FIGS. 18-20, this daily oral administration of HBS-101 at 2 mg/kg or 5 mg/kg produced a pronounced, dose-dependent inhibition of tumor growth compared with vehicle-treated mice, as evidenced by reduced tumor volumes over time, as shown in FIG. 18. Significantly lower terminal tumor weights were also observed, as shown in FIG. 19. Importantly, the subject mice maintained stable body weights throughout treatment, as shown in FIG. 20, indicating that HBS-101 achieves robust in vivo antitumor efficacy at well-tolerated doses. Tumor volume (FIG. 18), tumor weights (FIG. 19), body weights (FIG. 20) are shown. **p<0.0001; ****p<0.0001; ns, not significant. The nontreated tumor sample remains consistent in size whereas the tumor samples treated with 2 mg/kg HBS-101 and 5 mg/kg HBS-101, respectively, show a marked reduction in size. The reduction in tumor size is more pronounced at the higher 5 mg/kg HBS-101 dosage, as shown in FIG. 21, illustrating the dose-dependent antitumor activity reduction effect.

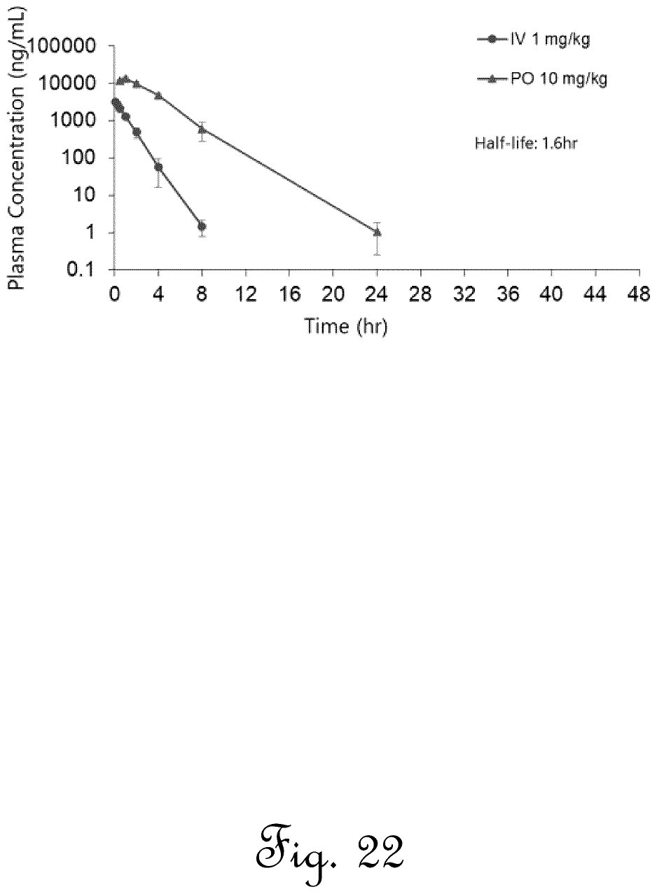

Favorable Pharmacokinetic Profile and Oral Exposure of HBS-101 in Rats: Turning now to FIG. 22, a graph illustrates the relationship between plasma concentration (ng/mL) over time (hr). Single-dose PK evaluation of HBS-101 in rats (1 mg/kg IV and 10 mg/kg oral) demonstrated robust (excellent) systemic exposure with a terminal plasma half-life of approximately 1.6 hours, as shown in FIG. 22. Both intravenous (IV) and oral routes produced sustained plasma concentrations within the therapeutically relevant range, supporting the feasibility of once- or twice-daily oral dosing for in vivo efficacy studies and eventual clinical use.

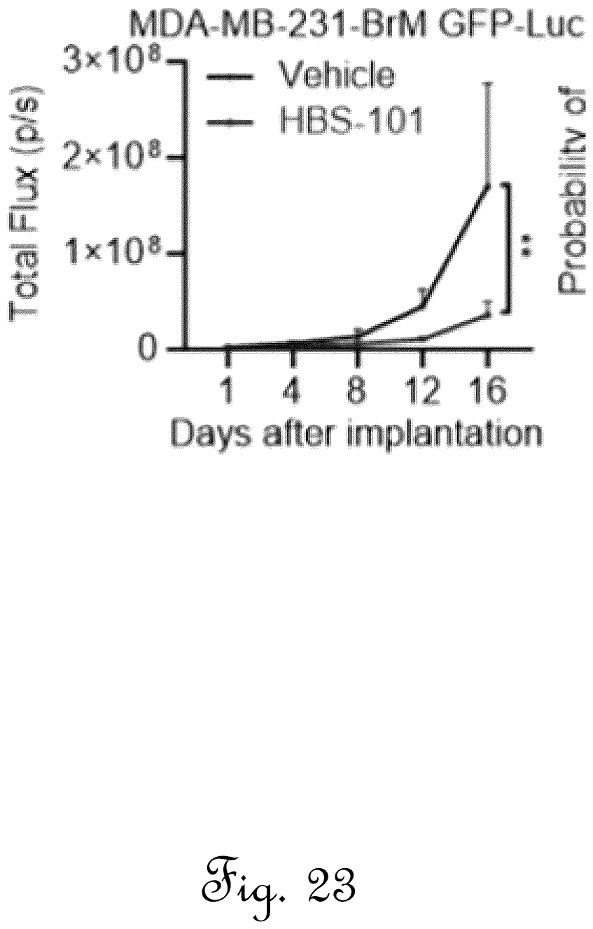

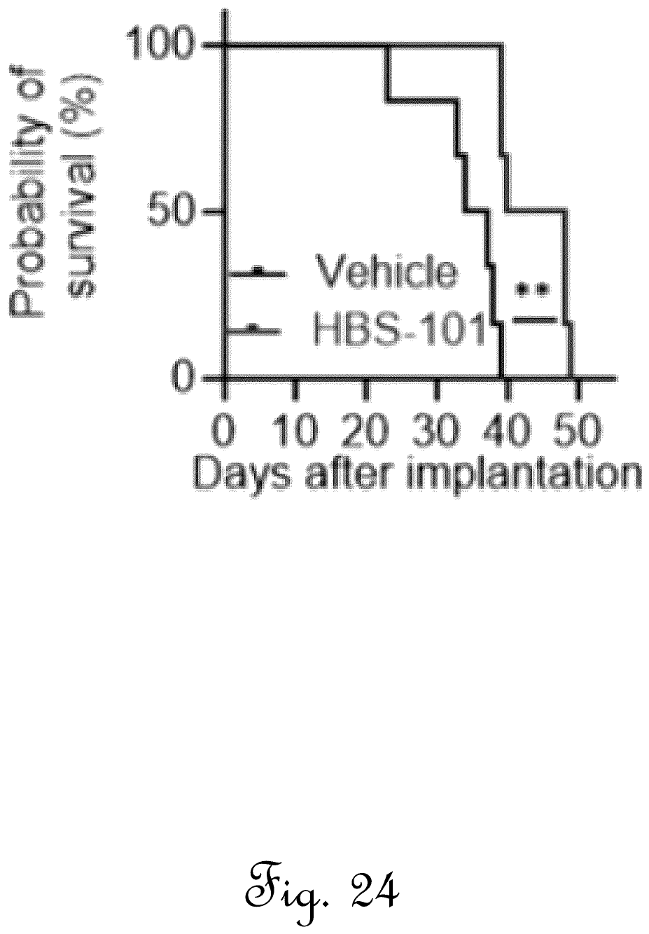

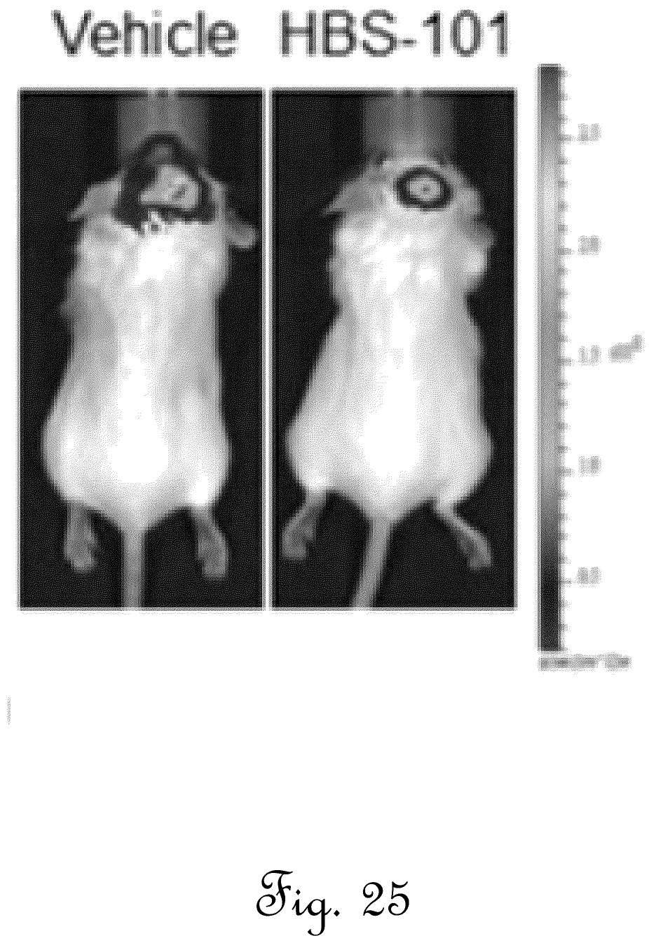

HBS-101 Suppresses Brain-Tropic TNBC Progression and Prolongs Survival in an Orthotopic Model: Referring now to FIGS. 23-25, in an orthotopic MDA-MB-231-BrM GFP-Luc model, MDA-MB-231-BrM cells were injected orthotopically, and mice were randomized to receive either vehicle of HBS-101 (10 mg/kg, i.p.) (n=6 mice/group). The intraperitoneal (i.p.) injection of mice treated with HBS-101 (10 mg/kg, i.p.) showed markedly reduced tumor burden compared with vehicle controls, as measured by bioluminescent signal over time (FIG. 23). HBS-101 treatment significantly extended overall survival (FIG. 24). FIG. 25 shows representative tumor images of the subject mice comparing the tumor treated with vehicle and the tumor treated with HBS-101. The representative tumor images, as shown in FIG. 25, are wholly consistent with the reduced intracranial tumor growth observed in representative in vivo imaging. These data demonstrate that HBS-101 is capable of controlling highly aggressive, brain-tropic TNBC and improving survival in vivo (**p<0.01).

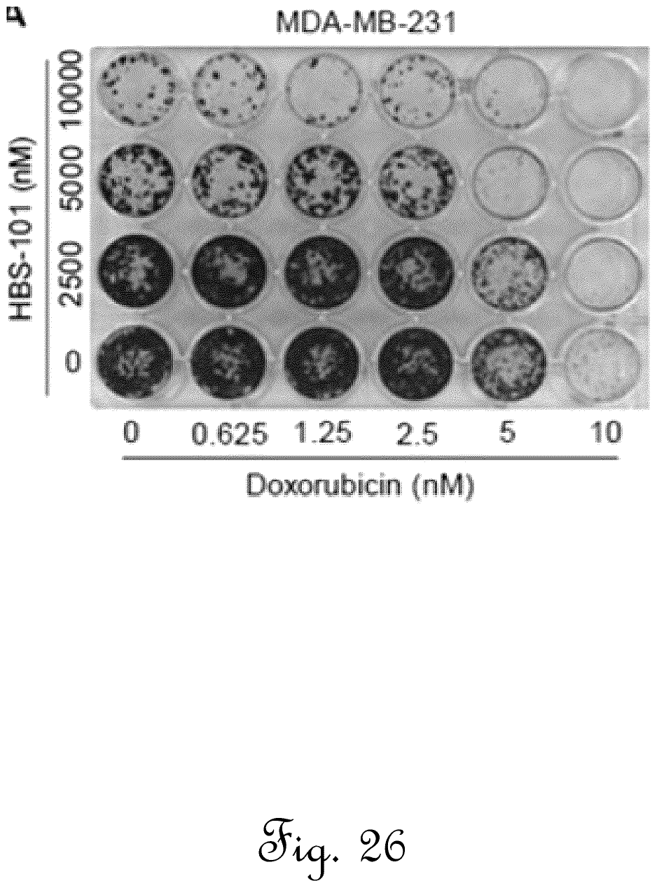

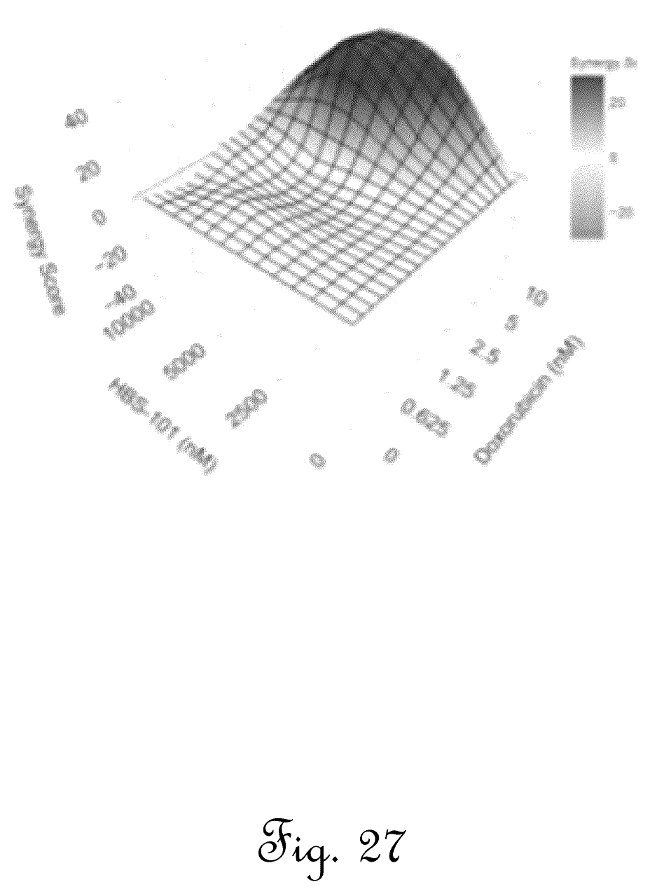

HBS-101 Acts Synergistically with Doxorubicin to Suppress TNBC Clonogenic Growth: Referring now to FIGS. 26-27, MDA-MB-231 cells were exposed to and treated with a matrix of either HBS-101 or doxorubicin or a combination of HBS-101 and doxorubicin concentrations for 5 days at various doses, followed by a 10-day colony formation assay. For example, and referring now to FIG. 26, the colonies formed after 10 days were stained with 0.5% crystal violet, scanned, and quantified using image J. Combination treatment produced a marked, dose-dependent loss of colonies that was greater than either agent alone across multiple dose pairs, as shown in FIG. 26.

Quantitative synergy analysis using a commercially available web-based and standalone software, here, SynergyFinder+, yielded a positive synergy score of 11.21 (p=3.72×10⁻¹¹), as shown in FIG. 27, confirming that HBS-101 and doxorubicin cooperate to enhance anti-tumor activity in TNBC cells. While present invention uses a web-based and standalone software commercially available as SynergyFinder+, other comparable software may also be used and still remain within the contemplation of the present invention.

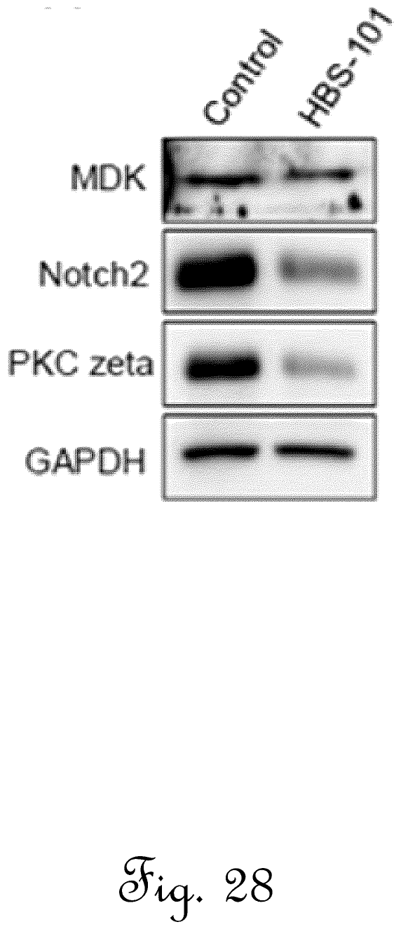

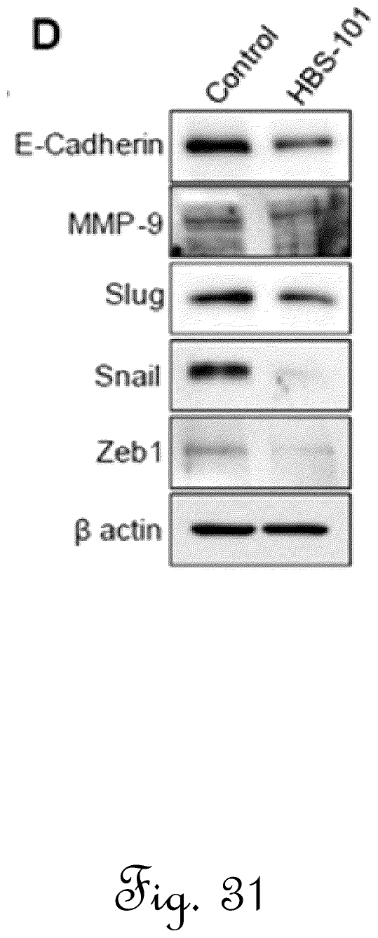

HBS-101 broadly suppresses MDK signaling and EMT programs in TNBC cells: Turning now to FIGS. 28-31, HCC-70 model cells were treated with HBS-101. More particularly, the HCC-70 model cells were treated with HBS-101 (20 PM, 20 h). Western blotting measured the treatment effect on expression of MDK receptors, known MDK target genes, MDK downstream signaling and EMT genes. **p, 0.0001; ****p<0.0001.

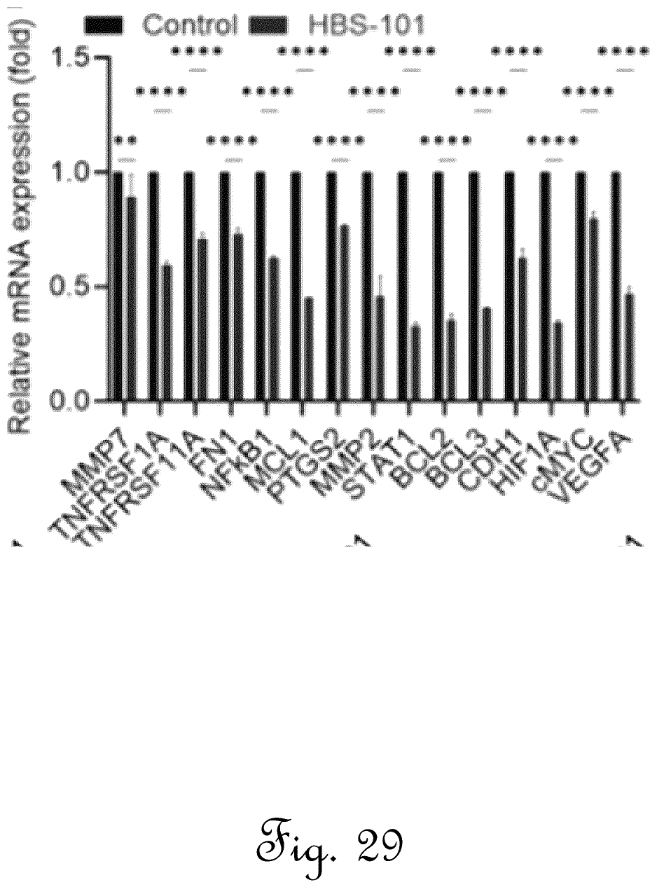

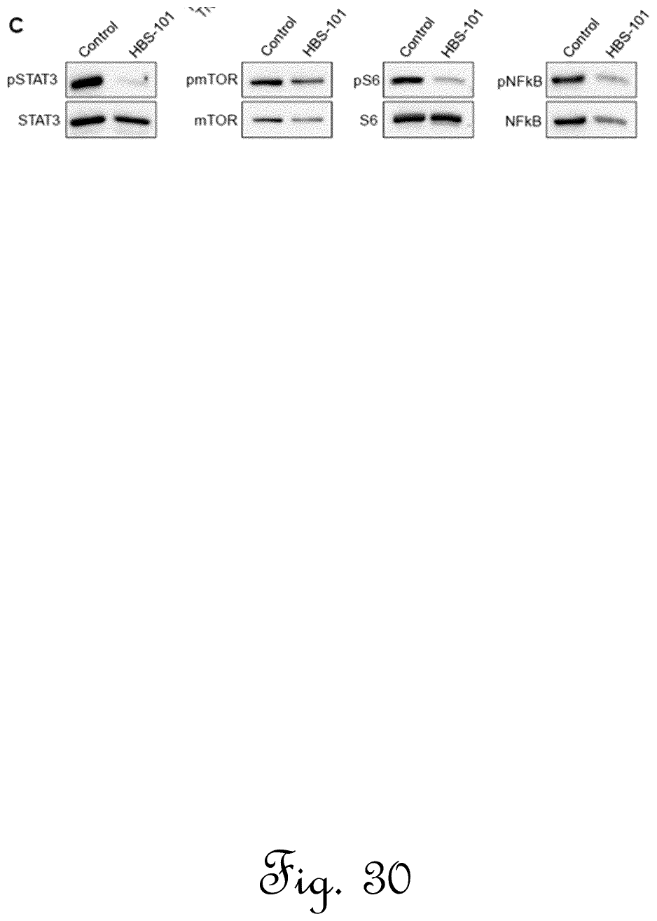

This treatment reduced MDK protein levels and decreased expression of key MDK receptors, including Notch2 and PKCζ, as shown in FIG. 28. This treatment also significantly downregulated a panel of MDK-responsive genes involved in invasion, survival, and inflammation, as shown in FIG. 29. Consistent with direct pathway inhibition, HBS-101 diminished activation of downstream effectors pSTAT3, pmTOR, pS6, and pNFκB, as shown in FIG. 30. HBS-101 also reversed EMT-associated changes, increasing E-cadherin while reducing MMP-9 and the transcription factors Slug, Snail, and Zeb1, as shown in FIG. 30, indicating that MDK blockade by HBS-101 shifts TNBC cells toward a less invasive, more epithelial phenotype.

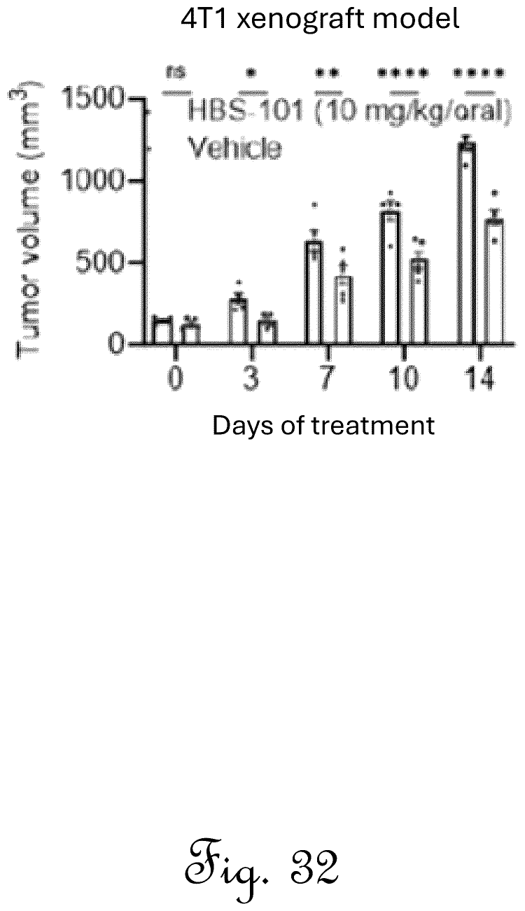

HBS-101 Inhibits Tumor Growth and Enhances NK-Cell Infiltration in an Immunocompetent TNBC Model: Referring now to FIGS. 32-35, in the syngeneic 4T1 TNBC xenograft model, the effect of HBS-101 on 4T1 TNBC xenograft tumor volume, body weight, tumor weight and status of NK cells are illustrated. *p<0.05; **p<0.01; ****p<0.0001; ns, not significant.

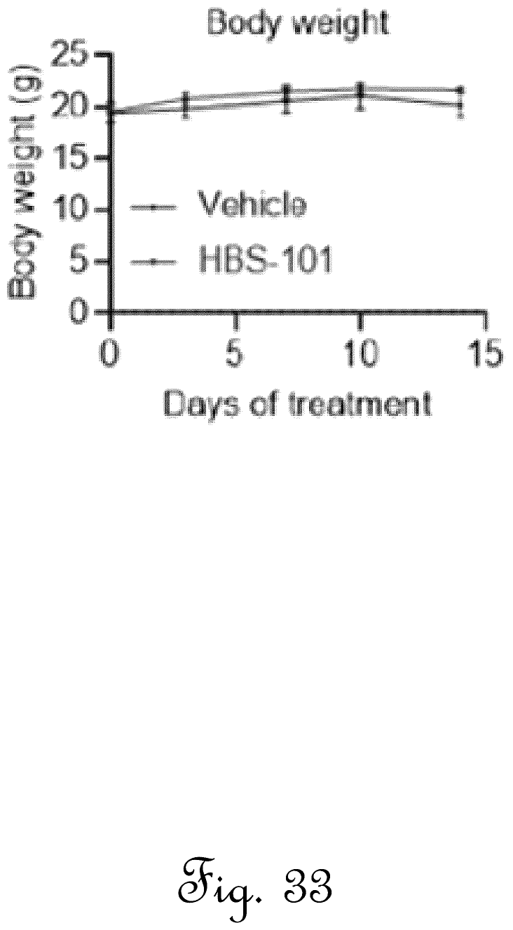

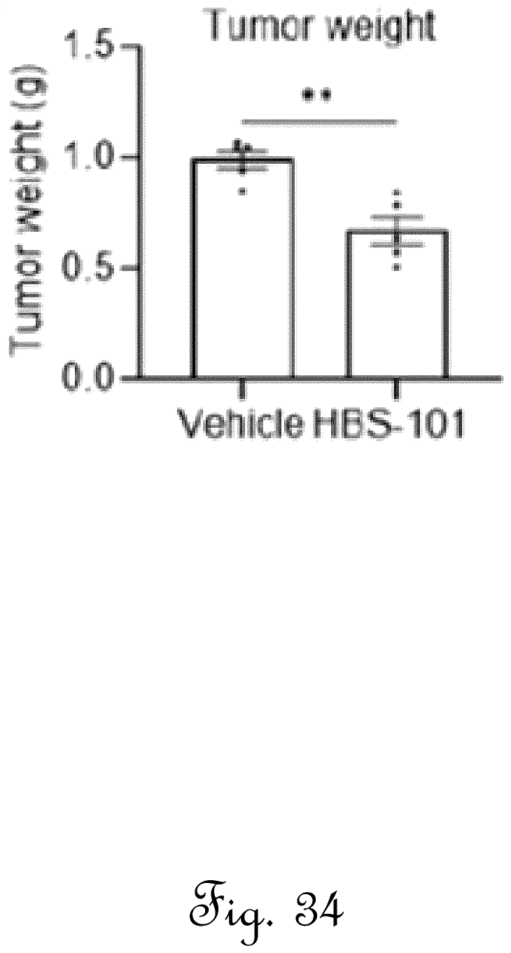

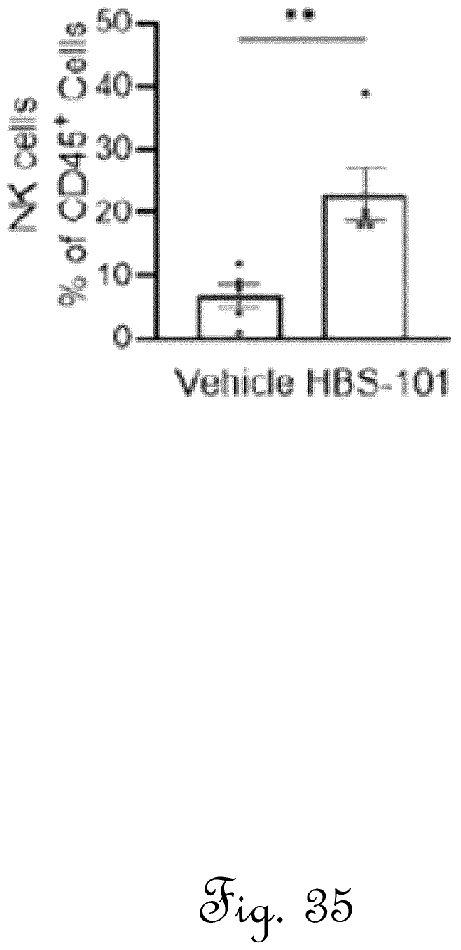

As shown in FIG. 32, oral administration of HBS-101 (10 mg/kg) significantly reduced tumor volume over a 14-day treatment period as compared with the vehicle. This led to a corresponding decrease in terminal tumor weight, as shown in FIG. 34, while body weights remained stable (FIG. 33) over the treatment period, indicating good tolerability. Flow-cytometric analysis of tumor-infiltrating leukocytes showed a significant increase in NK (NK1.1⁺) cells among CD45⁺ cells in HBS-101-treated tumors relative to controls, as shown in FIG. 35. These data demonstrate that HBS-101 exerts antitumor efficacy in an immunocompetent setting and favorably modulates the immune microenvironment by enhancing NK-cell infiltration.

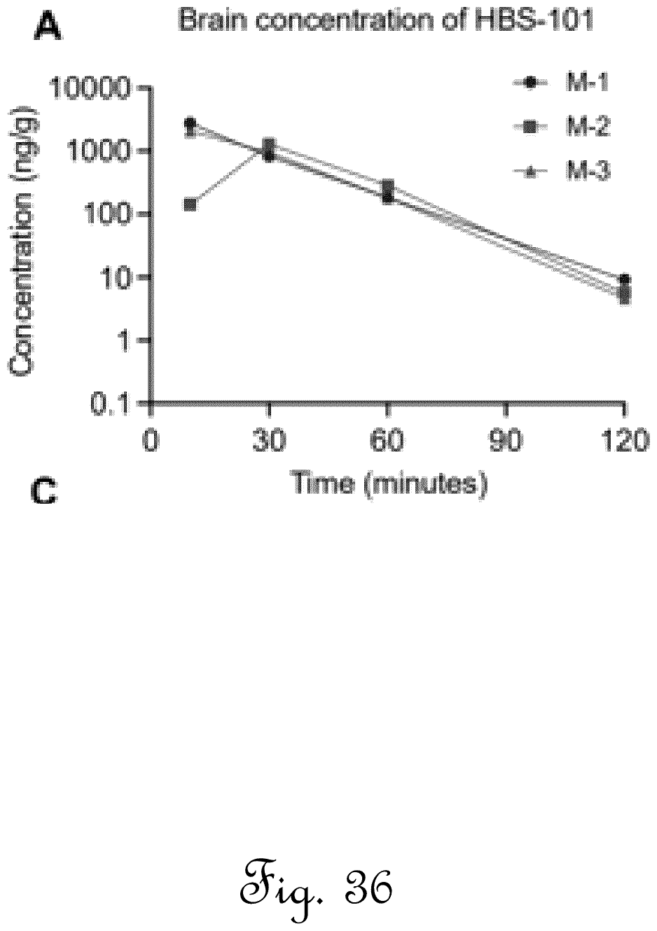

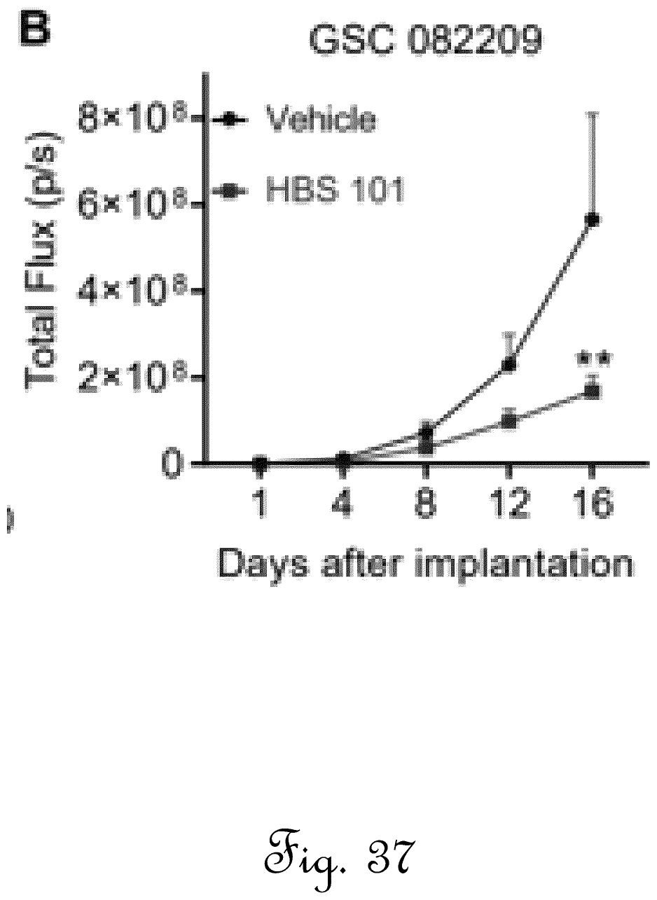

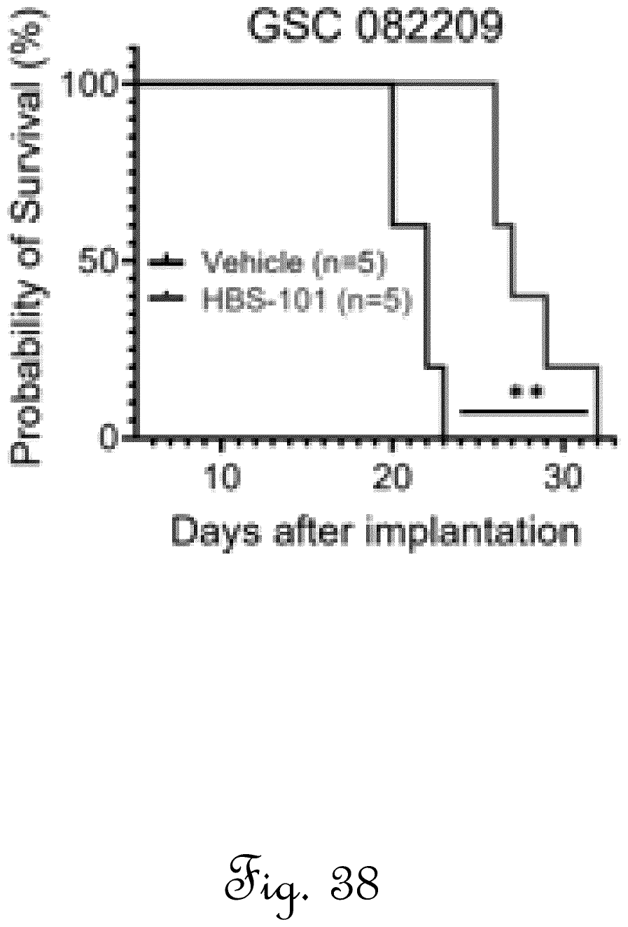

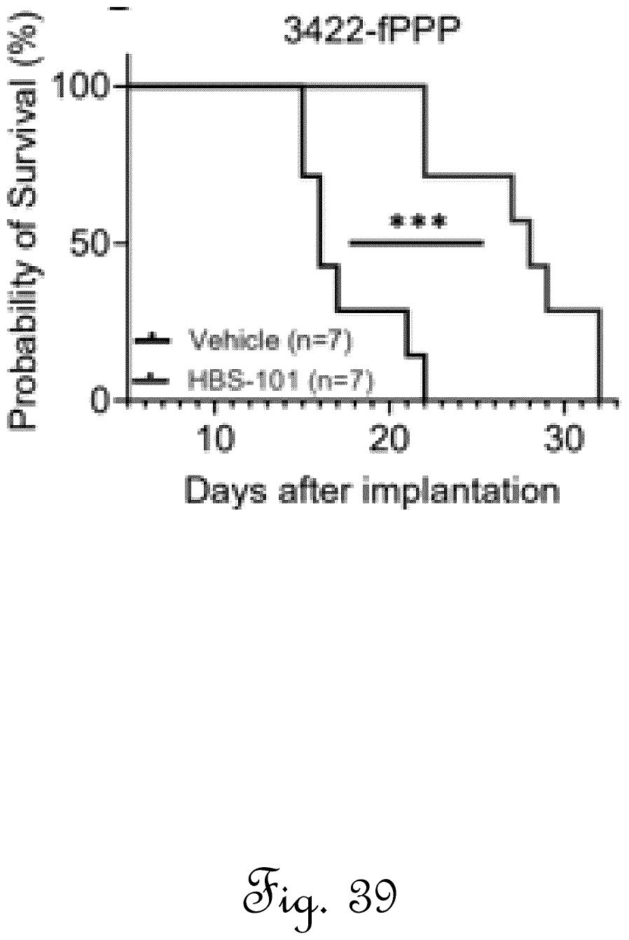

HBS-101 Penetrates the Brain and Prolongs Survival in Orthotopic Glioblastoma Models. Turning now to FIGS. 36-39, brain PK analysis showed that systemically administered HBS-101 rapidly achieved high concentrations in brain tissue and was maintained within a pharmacologically active range over time (FIG. 36). This confirms effective blood-brain barrier penetration. In an orthotopic patient-derived GSC 082209 glioblastoma model, and referring now to FIG. 37, HBS-101 treatment significantly reduced intracranial tumor burden as measured by bioluminescent flux. This led to a marked improvement in overall survival compared with vehicle-treated mice, as shown in FIG. 38 (**p<0.01). Similar survival benefit was observed in a second independent GBM model (3422-fPPP), where HBS-101 extended survival relative to control, as shown in FIG. 39 (***p<0.001). These data demonstrate HBS-101 is brain-penetrant and confers robust antitumor activity in aggressive glioblastoma models.

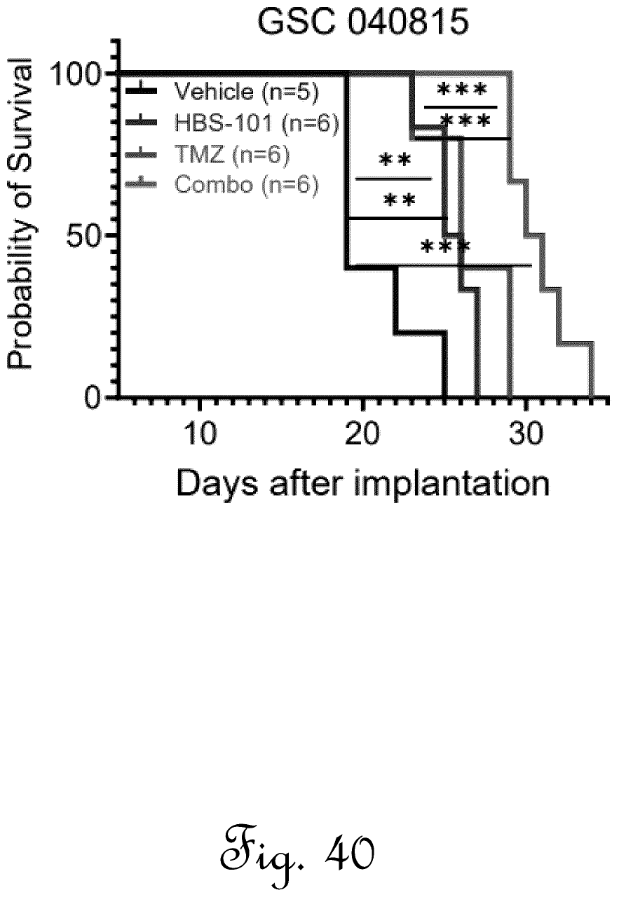

HBS-101 Enhances Temozolomide Efficacy and Extends Survival in an Orthotopic GBM Model. Referring now to FIG. 40, a graph of the probability of survival against days after implantation is shown of an orthotopic GSC 040815 glioblastoma model. In the orthotopic GSC 040815 glioblastoma model, treatment with HBS-101 alone or temozolomide (TMZ) alone significantly prolonged survival relative to vehicle controls. However, the synergistic effect of the combination of HBS-101 and TMZ provided a marked improvement over the effect of either alone. Notably, combined HBS-101+TMZ therapy produced the greatest survival benefit, shifting the Kaplan-Meier curve further to the right compared with either monotherapy, as shown in FIG. 40, indicating superior control of tumor progression and demonstrating that HBS-101 can potentiate the therapeutic effect of standard-of-care TMZ in vivo.

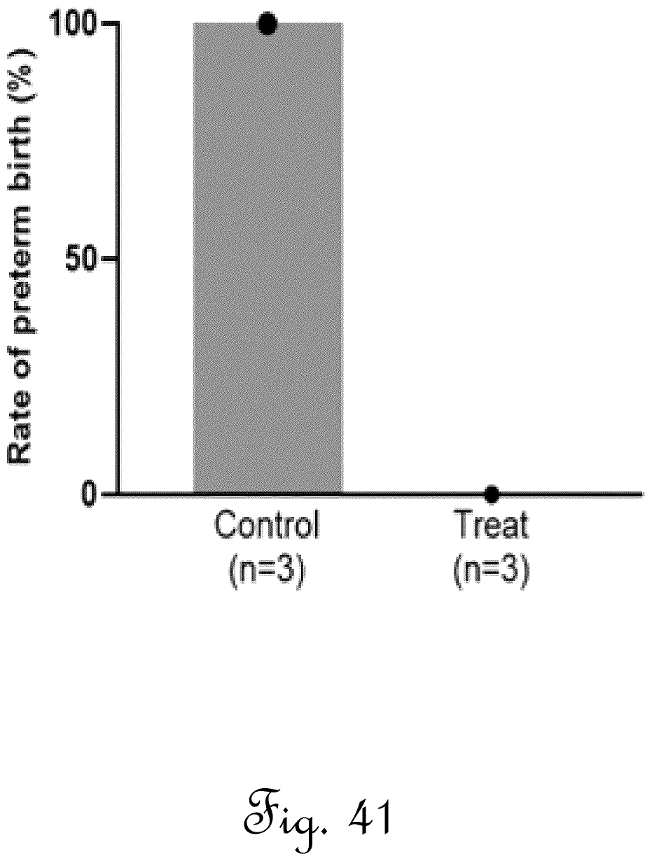

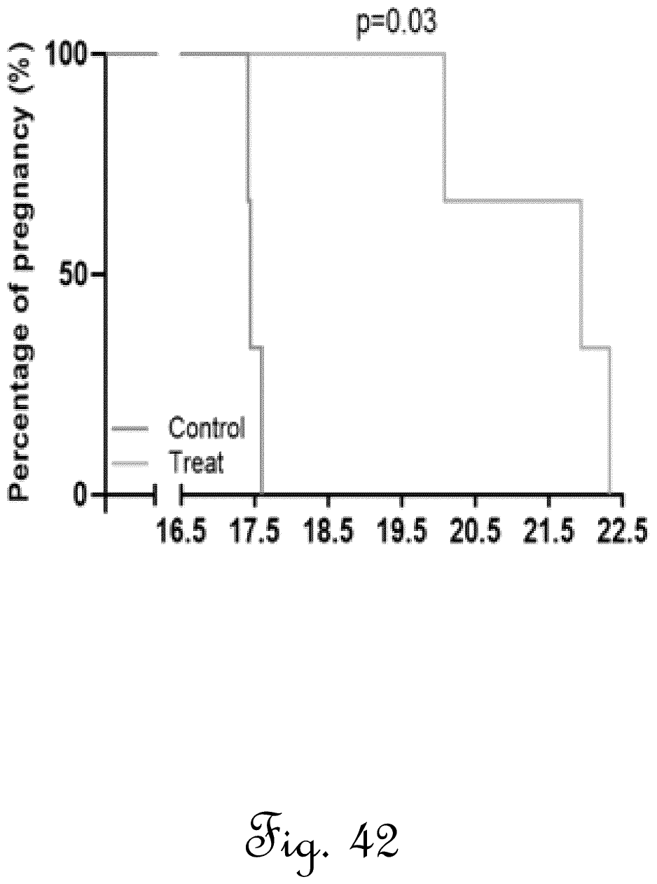

MDK-Targeted Treatment (HBS-353) Prolongs Gestation and Reduces Preterm Pregnancy Loss. Turning now to FIGS. 41 and 42, treatment with the MDK-modulating agent completely prevented preterm birth in this model. All control dams (3/3) experienced preterm delivery (FIG. 41), whereas none of the treated dams (0/3) delivered preterm, indicating a striking protective effect of the therapy against preterm birth. Referring now to FIG. 42, the animals receiving the MDK-modulating treatment-maintained pregnancy significantly longer than vehicle controls. All control dams experienced pregnancy loss by ~17.5 days post-coitum, whereas treated dams retained pregnancies out to ~22.5 days, with a rightward shift of the pregnancy-survival curve (p=0.03), as shown in FIG. 42. These data indicate that the treatment effectively delays preterm delivery and supports sustained pregnancy.

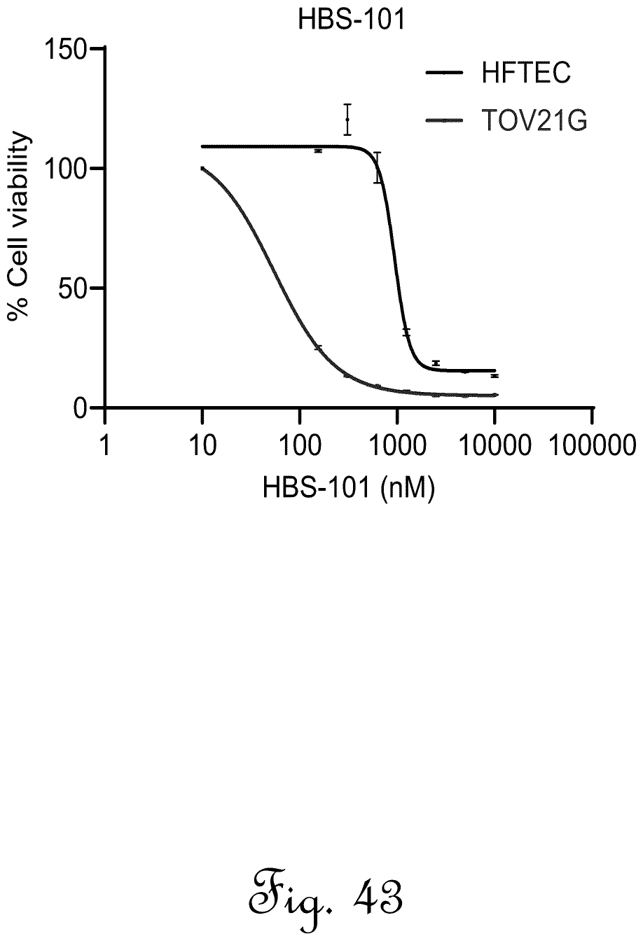

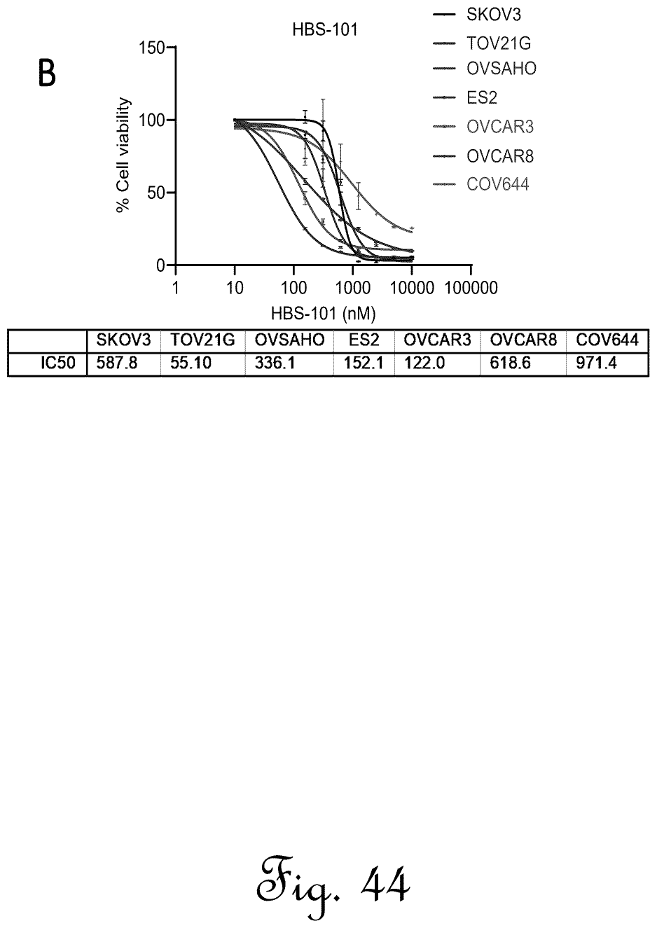

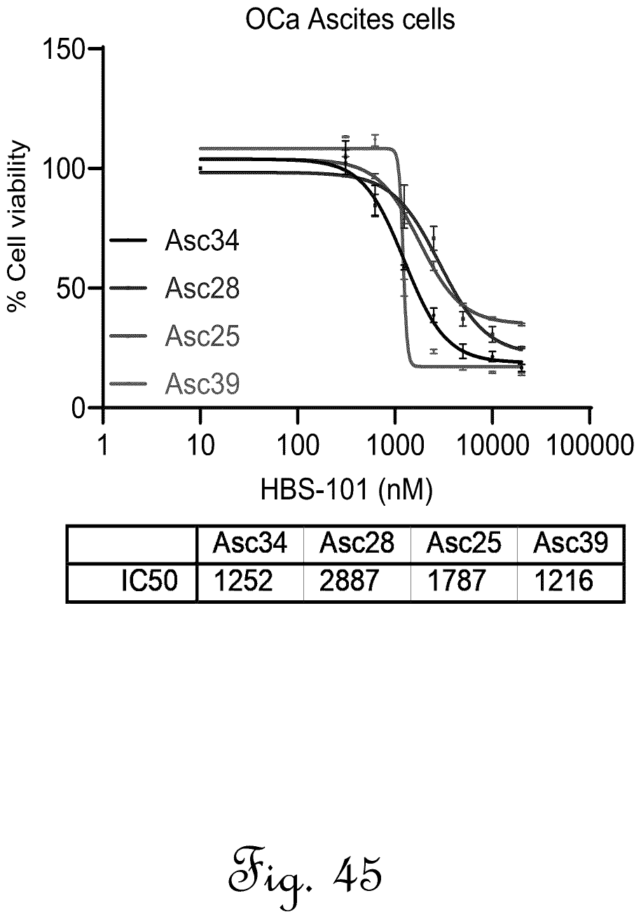

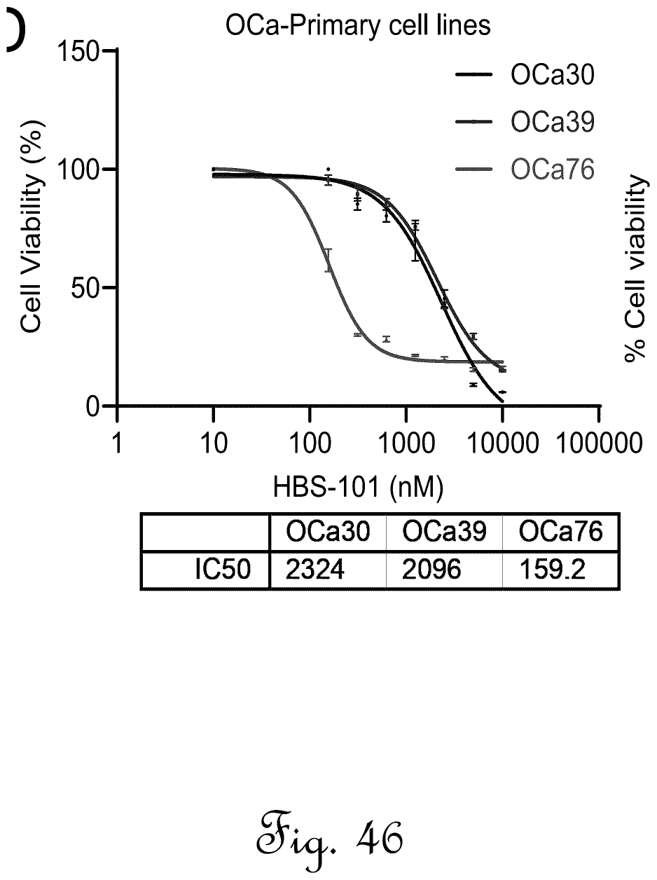

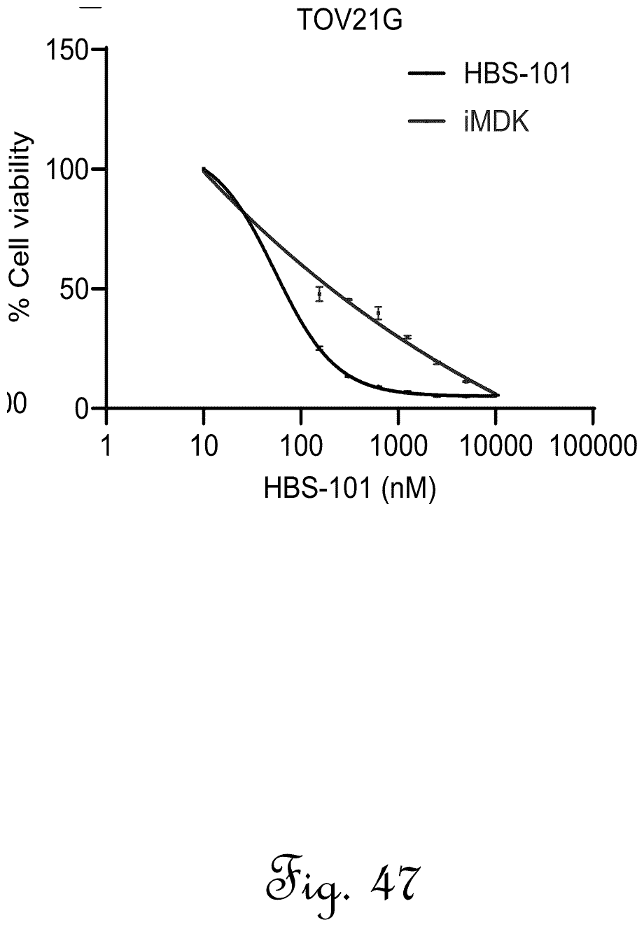

HBS-101 displays potent and selective activity across a spectrum of ovarian cancer models. Dose-response curves show that HBS-101 strongly reduces viability of the TOV21G ovarian cancer cell line while having markedly less effect on normal human fallopian tube epithelial cells (HFTEC), indicating tumor selectivity. The compound inhibits growth of multiple established ovarian cancer cell lines, patient-derived ascites tumor cells (Asc25/28/34/39), and 3D patient-derived ovarian cancer organoids (OCa30/39/76) with sub-to low-micromolar potency. FIG. 43 illustrates the effect of HBS-101 on the viability of normal HFTEC and OCa cell line TOV21G, and primary OCa cell lines as determined by the MTT assay. The effect of HBS-101 on the viability of established ovarian cancer cell lines, e.g., SKOV3, TOV21G, OVSAHO, ES2, OVCAR3, OVCAR8, COV644 (FIG. 44), ascites cells (FIG. 45), and primary OCa cells (FIG. 46) as determined by the MTT assay. Data are represented as mean±SEM. *p<0.05. When directly compared with the previously reported MDK modulator iMDK, and referring now to FIG. 47, HBS-101 achieves greater growth inhibition at lower concentrations, demonstrating superior efficacy as an MDK-targeted small-molecule agent.

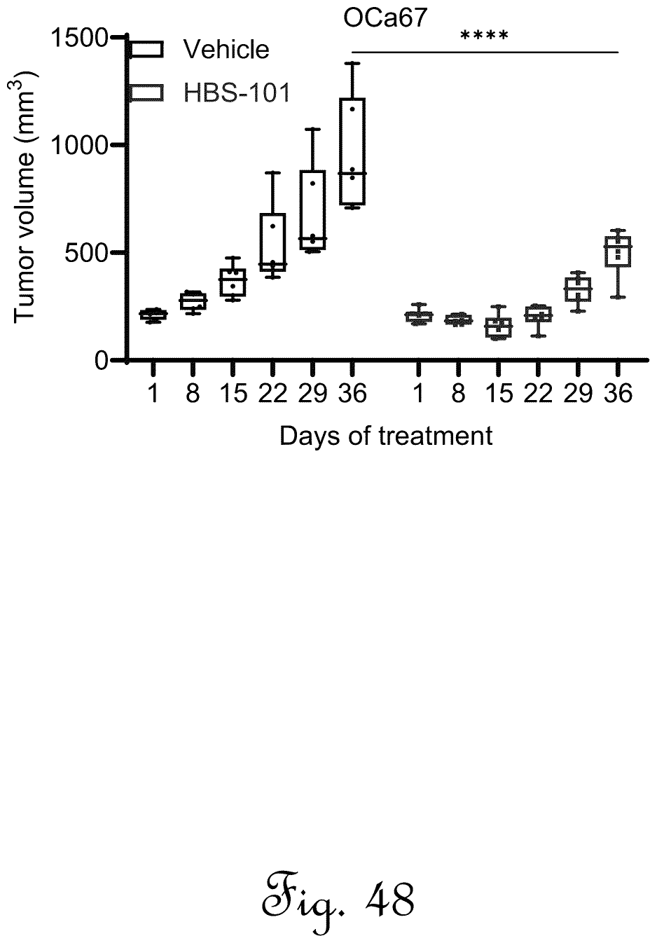

HBS-101 Suppresses Ovarian Tumor Growth In Vivo Without Affecting Body Weight. Referring now to FIGS. 48-50, in an ovarian cancer xenograft model, patient derived OCa-67 xenografts bearing mice were treated orally with vehicle or HBS-101 (10 mg/kg/day). Tumor volumes were measured twice a week.

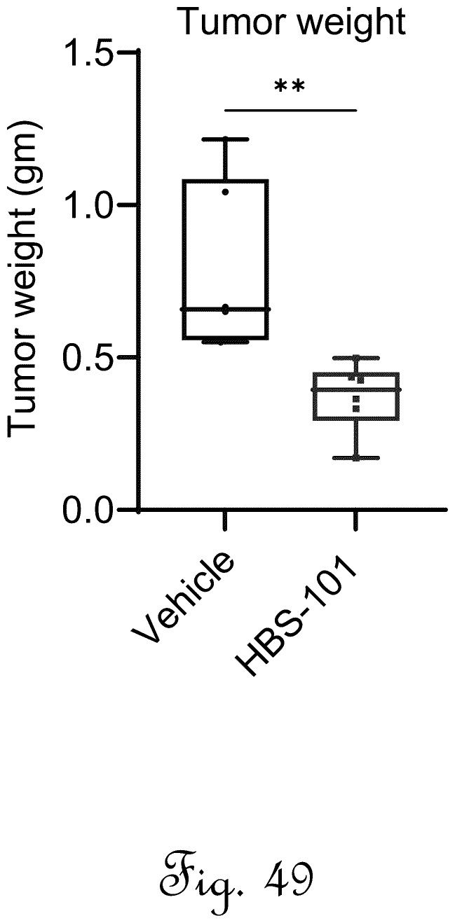

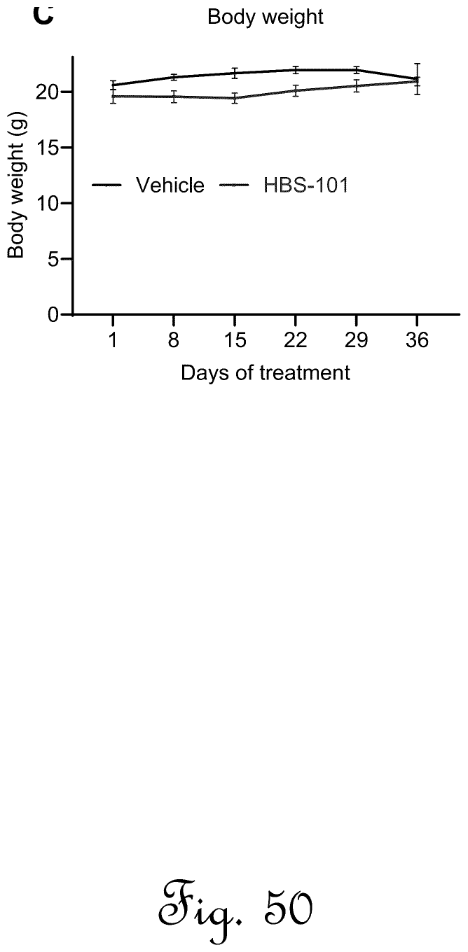

Treatment with HBS-101 produced a marked reduction in tumor burden compared with vehicle controls. Longitudinal measurements showed that tumor volumes in the HBS-101 group remained low throughout the dosing period, whereas vehicle-treated tumors expanded progressively, as shown in FIG. 48. At study end, tumors from HBS-101-treated animals were significantly lighter than those from control mice, as shown in FIG. 49, confirming a robust antitumor effect. Importantly, body weights were comparable between groups over the course of treatment, as shown in FIG. 50, indicating that HBS-101 was well tolerated at the tested regimen. **p<0.0001; ****p<0.0001.

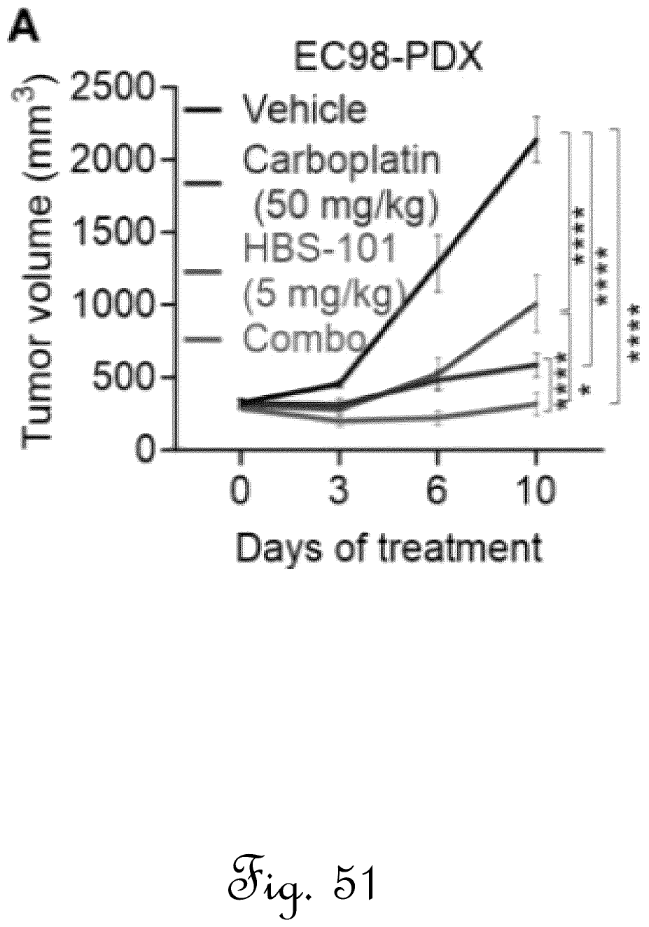

HBS-101 Synergizes with Carboplatin to Suppress Endometrial Cancer PDX Tumor Growth. Referring now to FIGS. 51-53, in an EC98 endometrial cancer PDX model, EC98-PDX (n=6) tumors in severe combined immunodeficiency (SCID) mice were treated with vehicle or HBS-101 (5 mg/kg, 5 days/week) or carboplatin (50 mg/kg—1 dose/week) or in combination. Tumor volume and weight of the vehicle and the treated tumors were measured. Body weights of treated mice were shown, as discussed below. **p<0.01; ****p<0.0001.

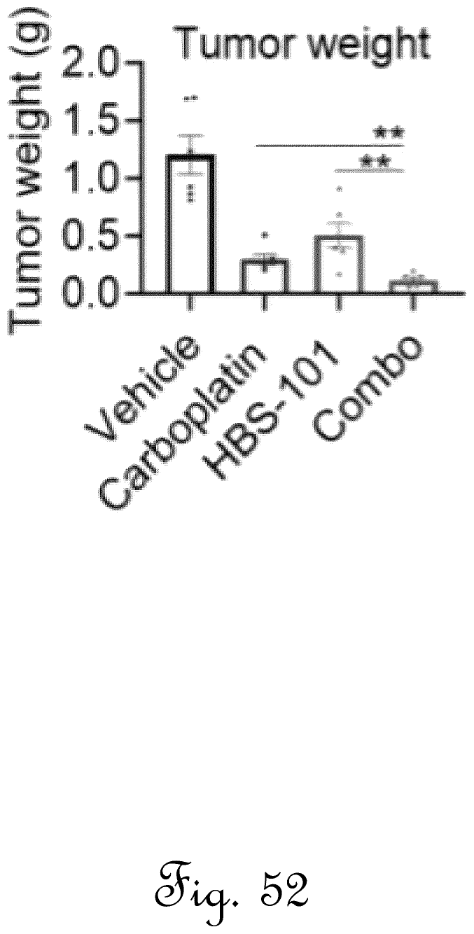

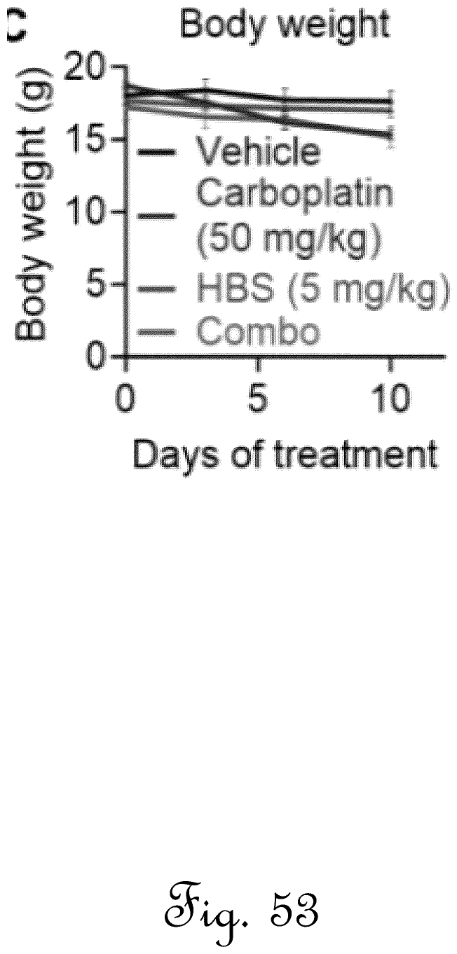

In the EC98 endometrial cancer PDX model, HBS-101 (5 mg/kg, 5 days/week) and carboplatin (50 mg/kg, once weekly) each reduced tumor growth relative to vehicle. However, the combination regimen produced the greatest antitumor effect, yielding the lowest tumor volumes over time, as shown in FIG. 51, and the smallest terminal tumor weights, as shown in FIG. 52. Importantly, and referring now to FIG. 53, combination-treated mice maintained stable body weight comparable to single-agent and vehicle groups, indicating that the enhanced efficacy was achieved without overt systemic toxicity. These results demonstrate that HBS-101 can be safely combined with standard-of-care carboplatin to achieve superior control of endometrial tumors.

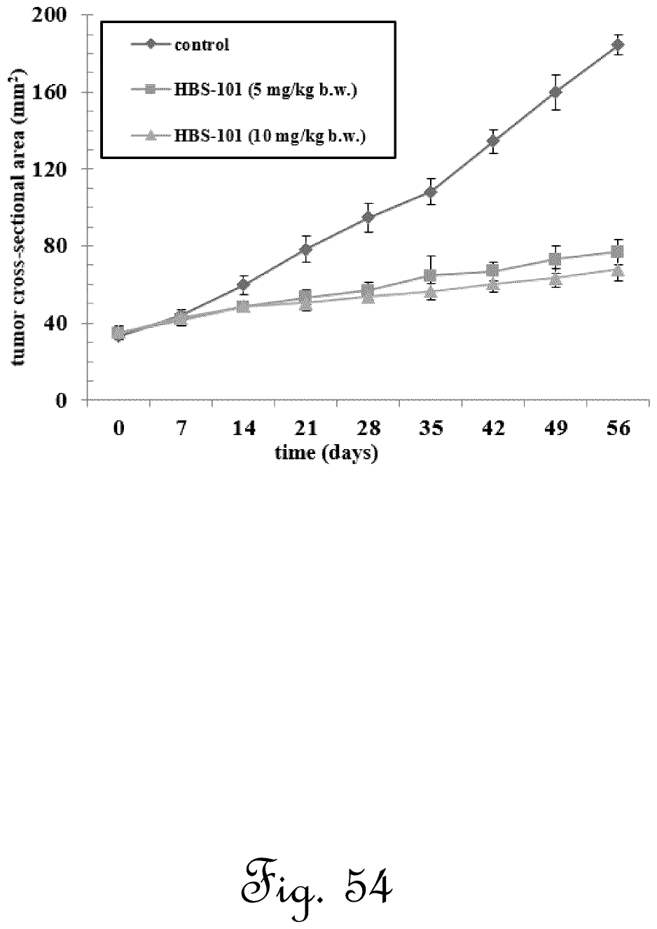

HBS-101 Inhibits Growth of SW1573 Non-Small Cell Lung Cancer Xenografts. Turning now to FIG. 54, a graph of tumor cross-sectional area (mm²) over time (days) is shown. In a SW1573 NSCLC xenograft model, systemic administration of HBS-101 markedly slowed tumor progression compared with vehicle-treated controls, as evidenced by reduced tumor growth over time and smaller endpoint tumor burdens in treated mice. Throughout the dosing period, animal body weights remained stable, indicating that the antitumor activity of HBS-101 was achieved without overt systemic toxicity. Consistent with the results previously discussed (See FIGS. 18-21), the reduction in tumor size (here, tumor cross-sectional area) is more pronounced at the higher 10 mg/kg HBS-101 dosage than at the lower 5 mg/kg HBS-101 dosage, as shown in FIG. 54, further illustrating the dose-dependent antitumor activity reduction effect.

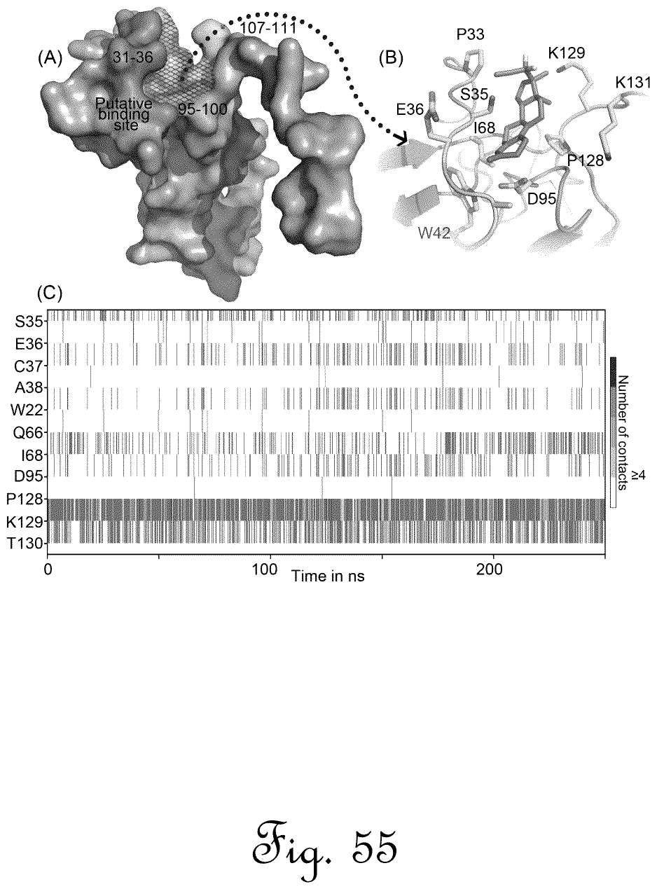

Modeled structure of MDK highlighting the putative binding site. Turning now to FIG. 55, a modeled structure of MDK is shown. The binding site of MDK is composed of three distinct amino acid stretches: residues 31-36 (Stretch 1, N-terminal), residues 95-100 (Stretch 2, C-terminal), and residues 107-111 (Stretch 3, C-terminal), as shown in FIG. 55 (showing three distinct amino acid stretches). (B) Close-up view of HBS-101 (displayed in pink sticks, i.e., pentagonal and hexagonal configurations lying in the region between P33, S35 and 168 on the one hand and K129, K131, P128 and D95 on the other) interaction with MDK (displayed in yellow sticks and cyan cartoon, i.e., P33, S35 and 168 and K129, K131, P128 and D95). The number of contacts maintained during 250 ns simulations can be shown in the Table charting number of contacts versus Time (ns), as shown in FIG. 55.

Biology tests. Several assays were performed, including cytotoxicity assays, soft agar colony formation assay, apoptosis assay, immunohistochemistry analysis, Western blotting, and tumor xenograft study. Each are described below:

Cytotoxicity assays. To identify the mechanism of action of midkine inhibitors, the cytotoxicity of these compounds in various cells lines were checked and derived IC50 values. Briefly, 5×103 cells were seeded in 96-well plates and incubated with compounds (0.0001-10 μmol/L) or dimethyl sulfoxide (DMSO; 0.02% v/v) for 24, 48, and 72 hours at 37° C. and cell viability was measured using a Fluoroscan plate reader (Nair et al, 2011).

Soft agar colony formation assay. Colonies of cancer cells formed soft agar in the presence and absence of the testing compounds is a standard assay to interpret in-vitro tumorigenic potential. The basal layer of agar was prepared by mixing 1% DNA grade agar melted and cooled to 40° C. with an equal volume of (2×) Dulbecco's Modified Eagle's Medium (DMEM) to obtain 0.5% agar that was dispersed in a 6-well plate and allowed to solidify. Although the present invention used Dulbecco's Modified Eagle's Medium (DMEM), other commercially available cell culture medium with comparable properties may also be used and still be within the contemplation of the present invention. A total of 0.6% agar was prepared in RPMI medium and mixed together with different types of cancer cells (0.5×106 cells/mL) and immediately plated on the basal layer in the presence or absence of testing compounds. The cultures were incubated at 37° C. in a CO₂ incubator for 2 weeks, and colonies were stained with 0.005% crystal violet and observed under a light microscope.

Apoptosis assay. Caspase-3/7 activity in HESE cells was measured using Caspase-Glo assay kit (commercially available under the brand PROMEGA®), as described before (Bhaskaran et al, 2013). Briefly, cells were homogenized in homogenization buffer (25 mmol/L HEPES, pH 7.5, 5 mmol/L MgCl2, and 1 mmol/L EGTA), protease inhibitors, and the homogenate was centrifuged at 13,000 rpm at 4° C. for 15 minutes. To 10 μL of the supernatant containing protein was added to an equal volume of the assay reagent and incubated at room temperature for 2 hours. The luminescence was measured using a luminometer. The treatment with midkine inhibitors induces apoptosis and reduces STAT3 phosphorylation.

Immunohistochemistry analysis. Patient derived tumor (melanoma) was treated with the compounds at 10 nM and 1 uM for 5 days in RPMI medium and harvested on day 6 and immunohistochemistry was performed for different antibodies including MDK, pSTAT3, Ki67.

Western blotting. In brief, different types of cancer cells such as MDA-MB-231/BT549 cells were treated with compounds for 3 days at different concentrations (10, 100 nM) and cell lysates were separated by 8% SDS-PAGE and transferred to polyvinylidene difluoride membranes. Membranes were then incubated with primary antibodies including phosphorylated and/or total pSTAT3. After overnight incubation at 4° C., membranes were incubated with secondary antibodies. Immunoreactive bands were then visualized by the enhanced chemiluminescence (ECL) detection system (GE healthcare).

Tumor xenograft study. Uniform suspensions of different cancer cell types such as triple negative breast cancer MDA-MB-231 cells or pancreatic ductal adenocarcinoma (PDAC) cells (2×10⁶) in 100 μL (0.02 carboxymethyl cellulose in phosphate buffered saline) were injected subcutaneously or orthotopically into the 4-5-week-old female athymic nude mice (provided by the Charles River Laboratories). After 10 days, when the tumor diameter reached 100 mm³, the mice were randomly allocated to 3 groups of each containing 6 animals. Group 1 served as the untreated control, Group 2 received midkine inhibitors orally at 2.5/5.0/10 mg/kg daily for 3 doses. All drug was formulated in a vehicle containing 30% captisol or 0.2% carboxy methyl cellulose (CMC). Tumors were allowed to reach palpability before drug intervention. Tumor size was measured every 3 days using digital Vernier Calipers and tumor volume was calculated using the ellipsoid formula [D×(d2)]/2, where D is the large diameter of the tumor and d represents the small diameter. On day 19, the mice were euthanized, and tumors were harvested for protein and gene expression studies since the control tumors reached maximum allowable size as allowed by the Institutional Animal Care and Use Committee (IACUC) regulations.

Synthesis of HBS-101 and analogues. The general formula (general formula I) for the midkine inhibitor (HBS-101) of the present invention is as follows:

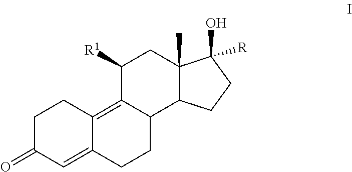

where R¹=hydrogen, alkyl, cycloalkyl, alkenyl, alkynyl, heteroalkyl, long chain fatty acid with at least one hetero atom in the chain (n=1 to 18).

Compounds having general formula I are synthesized as outlined in the following scheme (Scheme 1):

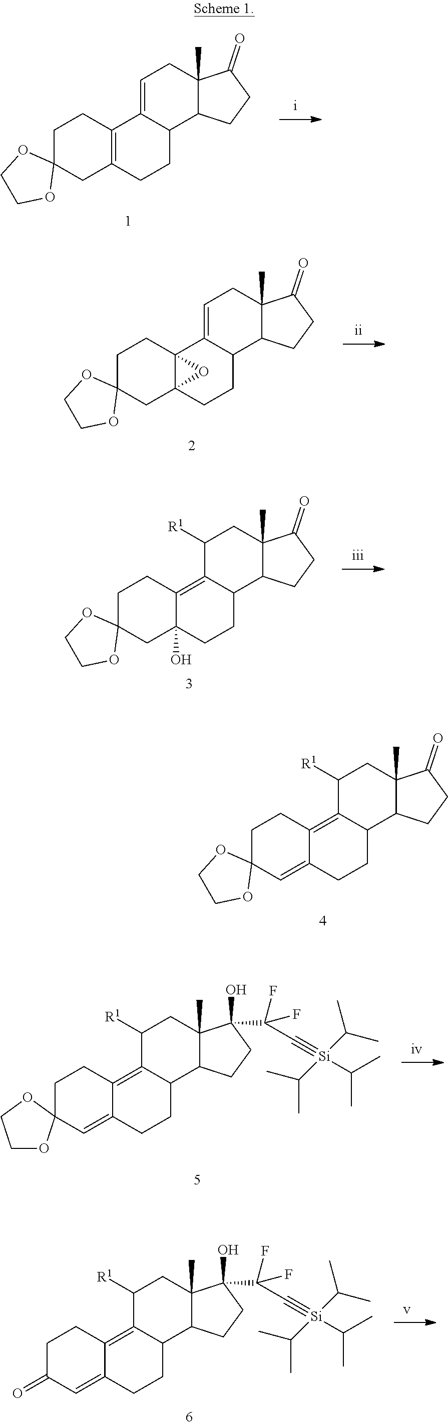

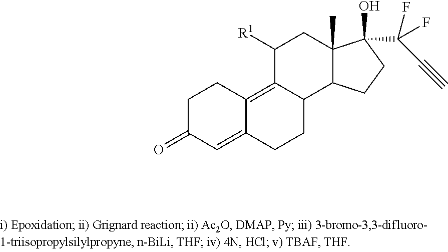

Scheme 2. Synthesis of HBS-101 starts from commercially available compound 1. 17-addition of 3-bromo, 3,3-difluoro-1-trisiopropylsilyl propyne in presence of n-butyl lithium affords Compound 2. The resulting compound was subsequently treated with hydrochloric acid and tetrabutylammonium fluoride to afford the final compound:

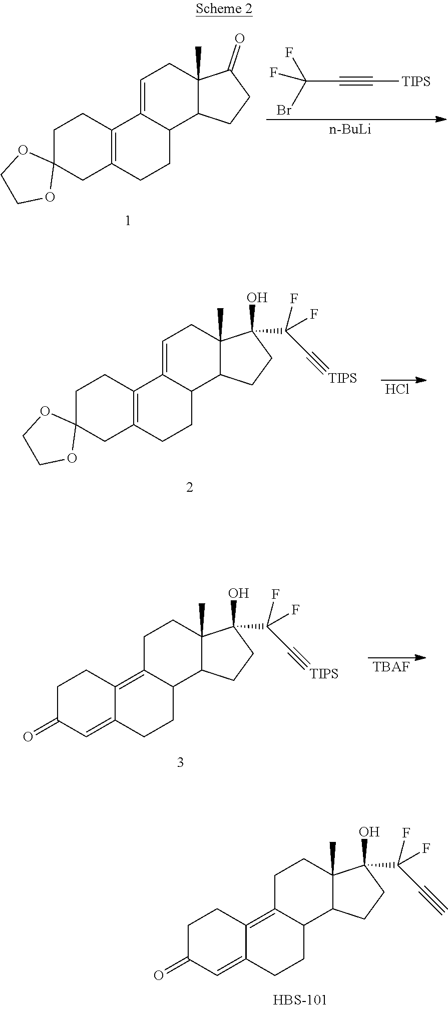

Scheme 3. Synthesis of HBS-353 starts from compound 4, which was prepared according to a reported procedure (EP 0700926 A1). Compound 4 was treated with n-butyl lithium and 3-bromo, 3,3-difluoro-1-trisiopropylsilyl propyne at −78° C. in THF to afford compound 5. Hydrolysis of the ketal group of compound 5 by 4N HCl afforded compound 6, which was treated with TBAF for removing the TIPS group to afford the final compound.

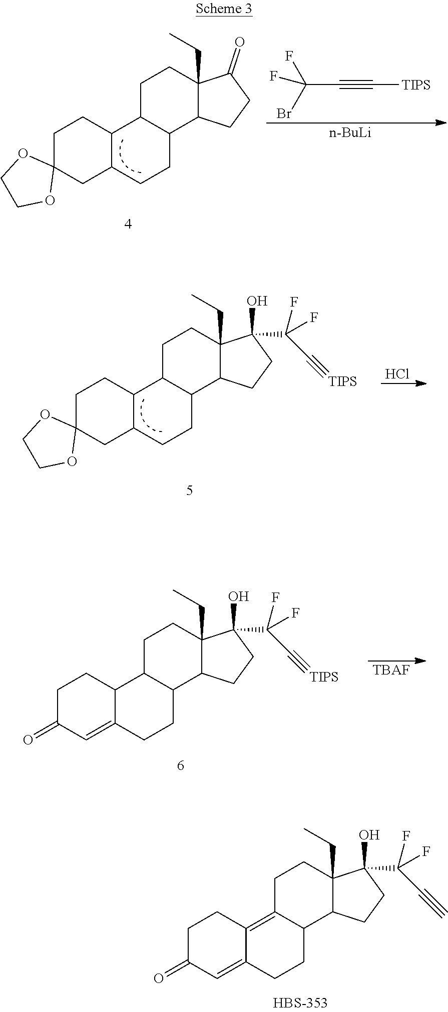

Experimental. All the reagents and solvents were analytical grade and used without further purification. Thin-layer chromatography (TLC) analyses were carried out on silica gel GF (Analtech) glass plates (2.5 cm×10 cm with 250 μM layer and pre-scored) and visualized by UV light (254 nm). Flash column chromatography was performed on 32-64 μM silica gel obtained from EM Science, Gibbstown, New Jersey. Melting points were determined on an Electro thermal MEL-TEMP apparatus and are uncorrected. Nuclear magnetic resonance spectra were recorded on a Bruker Avance II and AV (500 MHz and 300 MHz) spectrometer as deuterochloroform (CDCl₃) solutions using tetramethyl silane (TMS) as an internal standard (6=0) unless noted otherwise.

### 3,3-(ethylenedioxy)-17α-[1,1-difluoro-3-[tris(1-methylethyl)silyl]-2-propyn-1-yl]-17β-hydroxy-estra-5(10),9(11)-diene (2)

A solution of Compound 1 (lg, 3.18 mmol) and 3-bromo, 3,3-difluoro-1-trisiopropylsilyl propyne (2.97 g, 9.54 mmol) in 30 ml THF was cooled to −78° C. A 2.5 molar solution of n-BuLi (3.8 ml) in hexane was added dropwise and the internal temperature of the reaction was kept below −65° C. during the addition. The reaction mixture was stirred at −78° C. for 2.5 h. TLC showed complete conversion of starting material 1 to a nonpolar product. The reaction was quenched by adding saturated solution of NH₄Cl and extracted with ethyl acetate and the organic layer was washed with water and brine. The solvent was removed under vacuum to afford the crude product, which was purified by column chromatography (commercially available under the brand BIOTAGE®) on SiO₂ eluting with Hexane-Ethyl acetate solvent system to give 1.43 g of compound 2 in 82% yield.

¹H NMR (CDCl₃, 300 MHz) δ 0.91 (s, 3H), 1.06-1.11 (m, 21H), 1.19-1.30 (m, 1H), 1.42-1.52 (m, 1H), 1.72-2.53 (m, 16H), 2.71-2.75 (m, 1H), 3.88 (br m, 4H), 5.54-5.57 (m, 1H).

### 17α-[1,1-difluoro-3-[tris(1-methylethyl)silyl]-2-propyn-1-yl]-17β-hydroxy-estra-5(10),9(11)-diene (3)

A solution of Compound 2 (1.23 g, 2.25 mmol) in THF-MeOH (2:1, 30 ml) was refluxed with 4N HCl (2.25 ml, 9 mmol) for 5 h. HPLC showed complete conversion of starting material to a more polar compound. The reaction was cooled to room temperature and was quenched by the addition of sat. NaHCO₃. The reaction mixture was extracted with ethyl acetate and the combined organic layers were washed with water, brine and dried over anhydrous sodium sulfate. The solvent was removed under vacuum to afford the crude which was purified by column chromatography (BIOTAGE®) on SiO₂ eluting with Hexane-Ethyl acetate solvent system to give 830 mg of compound 3 in 73% yield.

¹H NMR (CDCl₃, 300 MHz) δ 1.04-1.11 (m, 24H), 1.24-1.28 (m, 1H), 1.68-1.99 (m, 6H), 2.10-2.17 (m, 1H), 2.28-2.47 (m, 6H), 2.82-2.94 (m, 2H), 5.67 (s, 1H).

### 17α-(1,1-difluoro-2-propyn-1-yl)-17β-hydroxy-estra-4,9-dien-3-one (HBS-101)

To a solution of 3 (800 mg, 1.59 mmol) in THF (20 ml) at r.t was added a 1M solution of tetrabutylammonium fluoride in THF (1.59 ml). The reaction was found to be complete in 30 minutes by HPLC analysis. The reaction was quenched by the addition of saturated ammonium chloride solution and was extracted with ethyl acetate. Combined organic layers were washed with water, brine and dried over anhydrous sodium sulfate. The solvent was removed under vacuum to afford the crude which was purified by column chromatography (BIOTAGE®) on SiO₂ eluting with Hexane-Ethyl acetate solvent system to give 520 mg of HBS-101 in 94% yield.

¹H NMR (CDCl₃, 500 MHz) δ 1.09 (s, 3H), 1.28-1.31 (m, 1H), 1.46-1.52 (m, 1H), 1.71-1.93 (m, 6H), 2.12-2.18 (m, 1H), 2.29-2.59 (m, 8H), 2.82-2.91 (m, 3H), 5.67 (s, 1H).

¹³C NMR (CDCl₃, 125 MHz) δ 14.69, 24.29, 25.78, 25.90, 27.50, 30.92, 32.90, 33.94, 37.02, 40.07, 47.12, 51.81, 85.54 (t, J=23 Hz), 122.22, 125.74, 145.35, 157.24, 199.96.

Compound 6. A solution of Compound 4 (2 g, 6.05 mmol) and 3-bromo, 3,3-difluoro-1-trisiopropylsilyl propyne (5.65 g, 18.15 mmol) in 60 ml THF was cooled to −78° C. A 2.5 molar solution of n-BuLi (7.3 ml) in hexane was added dropwise and the internal temperature of the reaction was kept below −65° C. during the addition. The reaction mixture was stirred at −78° C. for 2.5 h. TLC showed conversion of starting material 4 to a nonpolar product. The reaction was quenched by adding saturated solution of NH₄Cl and extracted with ethyl acetate and the organic layer was washed with water and brine. The solvent was removed under vacuum to afford the crude product 5, which was used as such for the next step.

The crude obtained (3.39 g, 6.05 mmol, theoretical yield amount) was dissolved in THF-MeOH (2:1, 60 ml) and was refluxed with 4N HCl (4.5 ml, 18.15 mmol) for 5 h. HPLC showed complete conversion of starting material to a more polar compound. The reaction was cooled to room temperature and was quenched by the addition of sat. NaHCO₃. The reaction mixture was extracted with ethyl acetate and the combined organic layers were washed with water, brine and dried over anhydrous sodium sulfate. The solvent was removed under vacuum to afford the crude which was purified by column chromatography (BIOTAGE®) on SiO₂ eluting with Hexane-Ethyl acetate solvent system to give 1.05 g of compound 6 in 33% yield.

¹H NMR (CDCl₃, 300 MHz) δ 0.99 (t J=9 Hz, 3H), 1.09-1.11 (m, 18H), 1.41-1.56 (m, 4H), 1.75-1.89 (m, 4H), 2.05-2.09 (m, 2H), 2.19-2.51 (m, 6H), 5.81 (br m, 1H).

### 13β-Ethyl-17α-(1,1-difluoro-2-propyn-1-yl)-17β-hydroxy-18-19-dinorpregn-4en-20y-yn-3-one (HBS-353)

To a solution of Compound 6 (lg, 1.94 mmol) in THF (20 ml) at r.t was added a 1M solution of tetrabutylammonium fluoride in THF (1.94 ml). The reaction was found to be complete in 30 minutes by HPLC analysis. The reaction was quenched by the addition of saturated ammonium chloride solution and was extracted with ethyl acetate. Combined organic layers were washed with water, brine and dried over anhydrous sodium sulfate. The solvent was removed under vacuum to afford the crude which was purified by column chromatography (BIOTAGE®) on SiO₂ eluting with Hexane-Ethyl acetate solvent system to give 675 mg of HBS-353 in 96% yield.

¹H NMR (CDCl₃, 500 MHz) δ 1.01 (t J=9 Hz, 3H), 1.07-1.16 (m, 2H), 1.41-1.49 (m, 4H), 1.75-1.93 (m, 4H), 2.20-2.52 (m, 6H), 2.85 (t, J=6 Hz, 1H), 5.81 (br m, 1H).

### 17β-3-Methoxy-17α-[1,1-difluoro-3-[tris(1-methylethyl)silyl]-2-propyn-1-yl]-androsta-3-5diene-17-ol (8)

A solution of Compound 7 (lg, 3.3 mmol) and 3-bromo, 3,3-difluoro-1-trisiopropylsilyl propyne (3.1 g, 9.9 mmol) in 30 ml THF was cooled to −78° C. A 2.5 molar solution of n-BuLi (3.96 ml) in hexane was added dropwise and the internal temperature of the reaction was kept below −65° C. during the addition. The reaction mixture was stirred at −78° C. for 2.5 h. TLC showed complete conversion of starting material 1 to a nonpolar product. The reaction was quenched by adding saturated solution of NH₄Cl and extracted with ethyl acetate and the organic layer was washed with water and brine. The solvent was removed under vacuum to afford the crude product, which was purified by column chromatography (BIOTAGE®) on SiO₂ eluting with Hexane-Ethyl acetate solvent system to give 1.2 g of compound 8 in 71% yield.

¹H NMR (CDCl₃, 300 MHz) δ 0.98 (m, 3H), 1.08-1.11 (m, 24H), 1.23-1.31 (m, 2H), 1.39-1.52 (m, 2H), 1.64-1.86 (m, 10H), 2.05-2.41 (m, 6H), 3.57 (s, 3H), 5.13 (brm, 1H), 5.23 (br m, 1H).

### 17α-[1,1-difluoro-3-[tris(1-methylethyl)silyl]-2-propyn-1-yl]-17β-hydroxy-androst-4-en-3-one (9)

A solution of Compound 8 (1.1 g, 2.06 mmol) in THF (20 ml) was cooled to 0° C. as 4N HCl (1.0 ml, 4.12 mmol) was added dropwise, and the reaction was stirred for 2 h warming to r.t. HPLC showed complete conversion of starting material to a more polar compound. The reaction was quenched by the addition of sat. NaHCO₃. The reaction mixture was extracted with ethyl acetate and the combined organic layers were washed with water, brine and dried over anhydrous sodium sulfate. The solvent was removed under vacuum to afford the crude which was purified by column chromatography (BIOTAGE®) on SiO₂ eluting with Hexane-Ethyl acetate solvent system to give 512 mg of compound 9 in 48% yield.

¹H NMR (CDCl₃, 500 MHz) δ 0.98 (s, 3H), 1.06-1.09 (m, 21H), 1.19 (s, 3H), 1.28-1.31 (m, 1H), 1.43-1.49 (m, 2H), 1.64-1.91 (m, 8H), 1.99-2.03 (m, 2H), 2.09-2.14 (m, 1H), 2.24-2.36 (m, 6H), 5.73 (s, 1H).

### 17α-(1,1-difluoro-2-propyn-1-yl)]-17β-hydroxy-androst-4-en-3-one (HBS 344)

To a solution of 9 (500 mg, 0.96 mmol) in THF (20 ml) at r.t was added a 1M solution of tetrabutylammonium fluoride in THF (0.96 ml). The reaction was found to be complete in 30 minutes by HPLC analysis. The reaction was quenched by the addition of saturated ammonium chloride solution and was extracted with ethyl acetate. Combined organic layers were washed with water, brine and dried over anhydrous sodium sulfate. The solvent was removed under vacuum to afford the crude which was purified by column chromatography (BIOTAGE®) on SiO₂ eluting with Hexane-Ethyl acetate solvent system to give 310 mg of HBS-344 in 89% yield.

¹H NMR (CDCl₃, 500 MHz) δ 0.99 (s, 3H), 1.19 (s, 3H), 1.28-1.31 (m, 1H), 1.43-1.49 (m, 2H), 1.61-1.89 (m, 8H), 2.01-2.04 (m, 1H), 2.24-2.43 (m, 1H), 2.85 (t, J=6 Hz, 1H), 5.73 (s, 1H).

The various embodiments described herein may be used singularly or in conjunction with other similar compounds. The present disclosure includes preferred or illustrative embodiments in which orally active, brain penetrant first-in-class small molecule midkine inhibitors for the treatment of malignancies and non-malignant diseases are described. Alternative embodiments of such midkine inhibitors and methods of synthesizing same can be used in carrying out the invention as claimed and such alternative embodiments are limited only by the claims themselves. Other aspects and advantages of the present invention may be obtained from a study of this disclosure and the drawings, along with the appended claims.

I claim:

1. Orally available small molecule (HBS-101 and its analogues) compounds for the treatment of various types of cancers that inhibit midkine (MDK or MK) and blocks the interaction to its receptors PKCzeta, Syndycans, Notch2, ALK and LRP1 or integrins and blocks downstream targets such as STAT3 and NFkB.

2. Orally available compounds, as recited in claim 1, to treat MDK overexpressing tumors (breast, endometrial, pancreatic, ovarian, liver, glioblastoma, medulloblastoma, lung cancer, and cortical tumors associated with neurofibromatosis (NF1-OPG) and non-malignant diseases involve inflammation including pericarditis, other cardiac inflammation, endometriosis, PCOS, uterine leiomyomas and preterm birth. MDK inhibitors abrogate fibrotic lesions in kidney and liver, blocks progression to carcinoma.

3. MDK inhibitor compounds, as recited in claim 2, inhibit lung inflammation and lung fibrosis. MDK small molecule inhibitor compounds that synergistically act with gemcitabine and other chemotherapeutic agents and immune checkpoint inhibitors.

4. Use of antiproliferative compounds of any one of claims 1 to 3 as a biomarker/companion diagnostic (CDx) for selecting suitable population to treat with MDK small molecule inhibitors to down-regulate phosphorylation of STAT3 and inhibit NFkB signaling.

5. Use of antiproliferative compounds of any one of claims 1 to 3 to block MDK action.

6. Midkine (MDK or MK) inhibitory compounds of any one of claims 1-5 having the general structure of 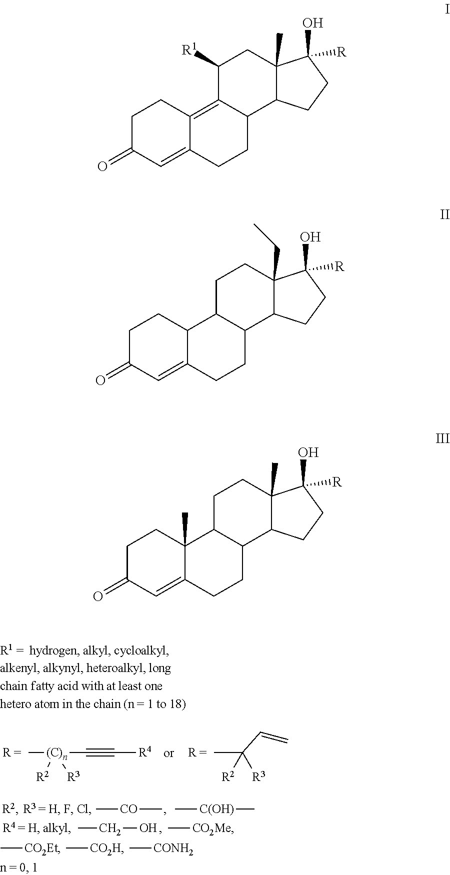
where R¹ is H, alkyl, cycloalkyl, alkenyl or a long chain fatty acid or any long chain with at least one hetero atom, and where R² and R³ are F and R⁴ is H, alkyl hydroxy alkyl, methyl or ethyl ester, acid or amide.

7. Specifically, the following midkine antagonists below were claimed:

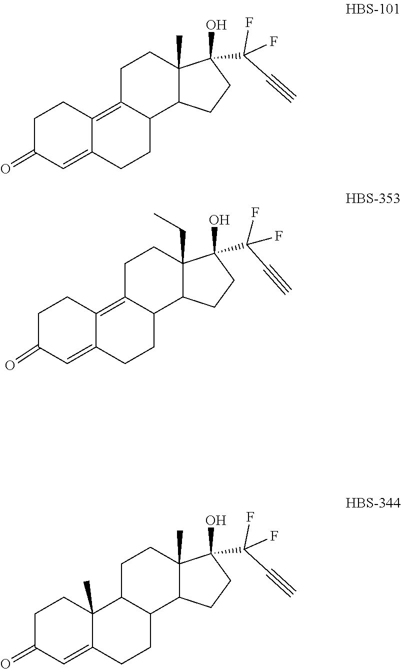 [SMILES: C#CC(F)(F)[C@]1(O)CCC2C3CCC4=CC(=O)CCC4=C3CC[C@@]21C ; C#CC(F)(F)[C@]1(O)CCC2C3CCC4=CC(=O)CCC4=C3CC[C@@]21CC ; C#CC(F)(F)[C@]1(O)CCC2C3CCC4=CC(=O)CC[C@]4(C)C3CC[C@@]21C]

8. A midkine inhibitor for the treatment of malignancies and non-malignant diseases, said midkine inhibitor having the following structure: 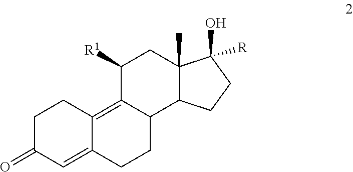
where R¹=hydrogen, alkyl, cycloalkyl, alkenyl, alkynyl, heteroalkyl, long chain fatty acid with at least one hetero atom in the chain (n=1 to 18), said midkine inhibitor configured to inhibit midkine and block the interaction to midkine-binding receptors and configured to block downstream targets, and wherein said midkine inhibitor is orally active and brain penetrant.

9. The midkine inhibitor for the treatment of malignancies and non-malignant diseases, as recited in claim 8, wherein said midkine-binding receptors comprise PKCzeta, Syndycans, Notch2, ALK and LRP1 or integrins.

10. The midkine inhibitor for the treatment of malignancies and non-malignant diseases, as recited in claim 9, wherein said downstream targets comprise STAT3 and NFkB.

11. The midkine inhibitor for the treatment of malignancies and non-malignant diseases, as recited in claim 10, wherein said malignancies comprise types of cancer including breast, endometrial, pancreatic, ovarian, liver, glioblastoma, medulloblastoma, lung cancer, and cortical tumors associated with neurofibromatosis.

12. The midkine inhibitor for the treatment of malignancies and non-malignant diseases, as recited in claim 11, wherein said nonmalignant diseases involve inflammation and comprise lung inflammation, lung fibrosis, pericarditis, other cardiac inflammation, endometriosis, PCOS, uterine leiomyomas and preterm birth.

13. The midkine inhibitor for the treatment of malignancies and non-malignant diseases, as recited in claim 12, wherein said midkine inhibitor abrogates fibrotic lesions in kidney and liver and blocks progression to carcinoma.

14. The midkine inhibitor for the treatment of malignancies and non-malignant diseases, as recited in claim 13, wherein said midkine inhibitor synergistically acts with gemcitabine and other chemotherapeutic agents and immune checkpoint inhibitors.

15. The midkine inhibitor for the treatment of malignancies and non-malignant diseases, as recited in claim 14, said midkine inhibitor having antiproliferative properties and used as a biomarker/companion diagnostic for selecting suitable population to treat with midkine inhibitors to down-regulate phosphorylation of STAT3 and inhibit NFkB signaling.

16. The midkine inhibitor for the treatment of malignancies and non-malignant diseases, as recited in claim 15, wherein said midkine inhibitor is HBS-101.

17. The midkine inhibitor for the treatment of malignancies and non-malignant diseases, as recited in claim 16, wherein said midkine inhibitor is an analogue of HBS-101.

18. The midkine inhibitor for the treatment of malignancies and non-malignant diseases, as recited in claim 17, wherein said analogue of HBS-101 is HBS-344.

19. The midkine inhibitor for the treatment of malignancies and non-malignant diseases, as recited in claim 17, wherein said analogue of HBS-101 is HBS-353.

20. A midkine inhibitory compound having the general structure of: 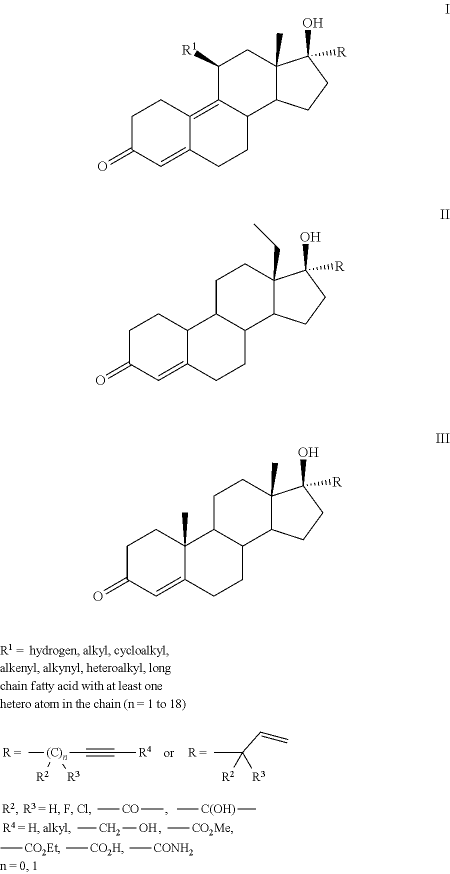
where R¹ is H, alkyl, cycloalkyl, alkenyl or a long chain fatty acid or any long chain with at least one hetero atom in the chain (n=1 to 18), and where R² and R³ are F and R⁴ is H, alkyl hydroxy alkyl, methyl or ethyl ester, acid or amide.

21. The midkine inhibitory compound of claim 21, having the structures below: 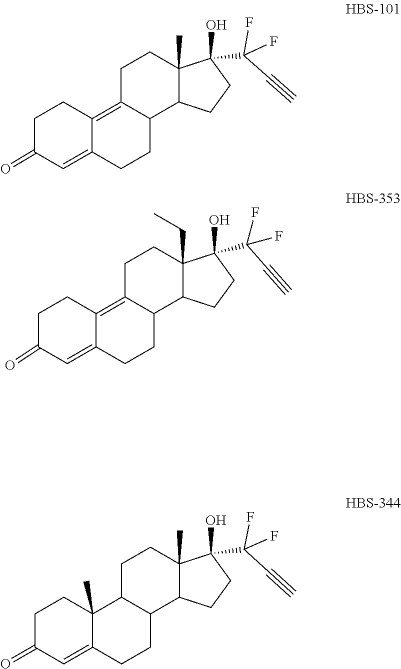 [SMILES: C#CC(F)(F)[C@]1(O)CCC2C3CCC4=CC(=O)CCC4=C3CC[C@@]21C ; C#CC(F)(F)[C@]1(O)CCC2C3CCC4=CC(=O)CCC4=C3CC[C@@]21CC ; C#CC(F)(F)[C@]1(O)CCC2C3CCC4=CC(=O)CC[C@]4(C)C3CC[C@@]21C]
